SEQUENCE LISTING (469 sequences)

SEQ ID NO: 1 - protein, 26 aa, Escherichia coli
MKIKTGARILALSALTTMMFSASALA

SEQ ID NO: 2 - protein, 25 aa, Escherichia coli
MMITLRKLPLAVAVAAGVMSAQAMA

SEQ ID NO: 3 - protein, 22 aa, Escherichia coli
MKYLLPTAAAGLLLLAAQPAMA

SEQ ID NO: 4 - protein, 23 aa, Escherichia coli
MKRNAKTIIAGMIALAISHTAMA

SEQ ID NO: 5 - protein, 18 aa, Escherichia coli
MRVLLFLLLSLFMLPAFS

SEQ ID NO: 6 - protein, 29 aa, Escherichia coli
MMNTRVWCKIIGMLALLVWLVSSPSVFAV

SEQ ID NO: 7 - protein, 19 aa, Escherichia coli
MKKIWLALAGLVLAFSASA

SEQ ID NO: 8 - protein, 26 aa, Escherichia coli
MKNRNRMIVNCVTASLMYYWSLPALA

SEQ ID NO: 9 - protein, 42 aa, Escherichia coli
MNNNDLFQASRRRFLAQLGGLTVAGMLGPSLLTPRRATAAQA

SEQ ID NO: 10 - protein, 21 aa, Escherichia coli
MKQSTIALALLPLLFTPVTKA

SEQ ID NO: 11 - protein, 21 aa, Escherichia coli
MKKTAIAIAVALAGFATVAQA

SEQ ID NO: 12 - protein, 19 aa, Klebsiella aerogenes
MLRYTCHALFLGSLVLLSG

SEQ ID NO: 13 - protein, 19 aa, Klebsiella pneumoniae
MLRYTRNALVLGSLVLLSG

SEQ ID NO: 14 - protein, 31 aa, Thermoanaerobacter ethanolicus
MFKRRTLGFLLSFLLIYTAVFGSMPVQFAKA

SEQ ID NO: 15 - protein, 31 aa, Thermoanaerobacter thermohydrosulfuricus
MFKRRALGFLLAFLLVFTAVFGSMPMEFAKA

SEQ ID NO: 16 - protein, 35 aa, Thermoanaerobacterium thermosulfurigenes
MNKKLFTNRFISFNMSLLLVLTAVFSSIPLHSVHA

SEQ ID NO: 17 - protein, 35 aa, Thermoanaerobacterium saccharolyticum
MYKKLFTKKFISFVMSLLLVLTAAFSSMPFHNVYA

SEQ ID NO: 18 - protein, 19 aa, Thermotoga maritima
MKTKLWLLLVLLLSALIFS

SEQ ID NO: 19 - protein, 23 aa, Escherichia coli
MKKNIAFLLASMFVFSIATNAYA

SEQ ID NO: 20 - protein, 21 aa, Escherichia coli
MKKSTLALVVMGIVASASVQA

SEQ ID NO: 21 - protein, 30 aa, Escherichia coli
MFSALRHRTAALALGVCFILPVHASSPKPG

SEQ ID NO: 22 - protein, 24 aa, Staphylococcus aureus
MKKLIFLIVIALVLSACNSNSSHA

SEQ ID NO: 23 - protein, 23 aa, Escherichia coli
MKNTIHINFAIFLIIANIIYSSA

SEQ ID NO: 24 - protein, 24 aa, Klebsiella oxytoca
MLKSSWRKTALMAAAAVPLLLASG

SEQ ID NO: 25 - protein, 43 aa, Bacillus cereus
MKNKKMLKIGMCVGILGLSITSLVTFTGGALQVEAKEKTGQVK

SEQ ID NO: 26 - protein, 20 aa, Escherichia coli
MKATKLVLGAVILGSTLLAG

SEQ ID NO: 27 - protein, 30 aa, Klebsiella oxytoca
MKRNRFFNTSAAIAISIALNTFFCSMQTIA

SEQ ID NO: 28 - protein, 24 aa, Escherichia coli
MNKNRGFTPLAVVLMLSGSLALTG

SEQ ID NO: 29 - protein, 23 aa, Escherichia coli
MTKHARFFLLPSFILISAALIAG

SEQ ID NO: 30 - protein, 29 aa, Escherichia coli
MKMRAVAVFTGMLTGVLSVAGLLSAGAYA

SEQ ID NO: 31 - protein, 26 aa, Escherichia coli
MNGSIRKMMRVTCGMLLMVMSGVSQA

SEQ ID NO: 32 - protein, 52 aa, Escherichia coli
MKRHLNTCYRLVWNHMTGAFVVASELARARGKRGGVAVALSLAAVTSLPVLA

SEQ ID NO: 33 - protein, 28 aa, Escherichia coli
MKTLKNMRRKNLCITLGLVSLLSRGANA

SEQ ID NO: 34 - protein, 24 aa, Escherichia coli
MLKKSILPMSCGVLVMVMSGLLDA

SEQ ID NO: 35 - protein, 21 aa, Escherichia coli
MKTSSFIIVILLCFRIENVIA

SEQ ID NO: 36 - protein, 22 aa, Escherichia coli
MKIRRIVSTIAIALSVFTFAHA

SEQ ID NO: 37 - protein, 22 aa, Escherichia coli
MNKTLIAAAVAGIVLLASNAQA

SEQ ID NO: 38 - protein, 49 aa, Escherichia coli
MNKAYSIIWSHSRQAWIVASELARGHGFVLAKNTLLVLAVVSTIGNAFA

SEQ ID NO: 39 - protein, 22 aa, Escherichia coli
MMYRIRNWLVATLLLLCTPVGA

SEQ ID NO: 40 - protein, 30 aa, Escherichia coli
MGSPSLYSARKTTLALAVALSFAWQAPVFA

SEQ ID NO: 41 - protein, 19 aa, Escherichia coli
MFKTTLCALLITASCSTFA

SEQ ID NO: 42 - protein, 17 aa, Escherichia coli
MKLAACFLTLLPGFAVA

SEQ ID NO: 43 - protein, 25 aa, Escherichia coli
MRKITQAISAVCLLFALNSSAVALA

SEQ ID NO: 44 - protein, 23 aa, Escherichia coli
MHKFTKALAAIGLAAVMSQSAMA

SEQ ID NO: 45 - protein, 19 aa, Escherichia coli
MKKVILSLALGTFGLGMAE

SEQ ID NO: 46 - protein, 22 aa, Escherichia coli
MKKSILALSLLVGLSTAASSYA

SEQ ID NO: 47 - protein, 19 aa, Escherichia coli
MKKVLIAALIAGFSLSATA

SEQ ID NO: 48 - protein, 19 aa, Escherichia coli
MKKLVLAALLASFTFGASA

SEQ ID NO: 49 - protein, 22 aa, Escherichia coli
MEFFKKTALAALVMGFSGAALA

SEQ ID NO: 50 - protein, 22 aa, Escherichia coli
MRRFLTTLMILLVVLVAGLSAL

SEQ ID NO: 51 - protein, 20 aa, Escherichia coli
MKWLCSVGIAVSLALQPALA

SEQ ID NO: 52 - protein, 21 aa, Escherichia coli
MRYIRLCIISLLATLPLAVHA

SEQ ID NO: 53 - protein, 23 aa, Escherichia coli
MSIQHFRVALIPFFAAFCLPVFA

SEQ ID NO: 54 - protein, 18 aa, Escherichia coli
MRLLPLVAAATAAFLVVA

SEQ ID NO: 55 - protein, 20 aa, Escherichia coli
MKTFAAYVITACLSSTALAS

SEQ ID NO: 56 - protein, 24 aa, Escherichia coli
MNLKKIAIASSVFAGITMALTCHA

SEQ ID NO: 57 - protein, 20 aa, Escherichia coli
MIKKASLLTACSVTAFSAWA

SEQ ID NO: 58 - protein, 22 aa, Escherichia coli
MRKSLLAILAVSSLVFSSASFA

SEQ ID NO: 59 - protein, 25 aa, Escherichia coli
MEALGMIETRGLVALIEASDAMVKA

SEQ ID NO: 60 - protein, 18 aa, Escherichia coli
MRFLLGVLMLMISGSALA

SEQ ID NO: 61 - protein, 19 aa, Escherichia coli
MHKLFYLLSLLMAPFVANA

SEQ ID NO: 62 - protein, 22 aa, Escherichia coli
MKHKKKNRLVVAISVALIPYIG

SEQ ID NO: 63 - protein, 25 aa, Escherichia coli
MFRLNPFVRVGLCLSAISCAWPVLA

SEQ ID NO: 64 - protein, 34 aa, Escherichia coli
MSKRNAVTTFFTNRVTKALGMTLALMMTCQSAMA

SEQ ID NO: 65 - protein, 19 aa, Escherichia coli
MIKFSATLLATLIAASVNA

SEQ ID NO: 66 - protein, 20 aa, Escherichia coli
MKSKLIILLMLVPFSSFSTE

SEQ ID NO: 67 - protein, 20 aa, Escherichia coli
MKSKLIILLTLVPFSSFSTG

SEQ ID NO: 68 - protein, 20 aa, Escherichia coli
MKLLKVAAIAAIVFSGSALA

SEQ ID NO: 69 - protein, 21 aa, Escherichia coli
MKNKLLFMMLTILGAPGIAAA

SEQ ID NO: 70 - protein, 17 aa, Escherichia coli
MNTLLLLAALSSQITFN

SEQ ID NO: 71 - protein, 22 aa, Escherichia coli
MKRYLRWIVAAEFLFAAGNLHA

SEQ ID NO: 72 - protein, 19 aa, Escherichia coli
MRVKHAVVLLMLISPLSWA

SEQ ID NO: 73 - protein, 15 aa, Escherichia coli
MQRLFLLVAVMLLSG

SEQ ID NO: 74 - protein, 17 aa, Escherichia coli
MRKLFLSLLMIPFVAKA

SEQ ID NO: 75 - protein, 20 aa, Escherichia coli
MVKKAIVTAMAVISLFTLMG

SEQ ID NO: 76 - protein, 24 aa, Escherichia coli
MRLRKYNKSLGWLSLFAGTVLLSG

SEQ ID NO: 77 - protein, 24 aa, Escherichia coli
MFKSTLAAMAAVFALSALSPAAMA

SEQ ID NO: 78 - protein, 25 aa, Escherichia coli
MAVNLLKKNSLALVASLLLAGHVQA

SEQ ID NO: 79 - protein, 29 aa, Escherichia coli
MNTIFSARIMKRLALTTALCTAFISAAHA

SEQ ID NO: 80 - protein, 27 aa, Escherichia coli
MTQYSSLLRGLAAGSAFLFLFAPTAFA

SEQ ID NO: 81 - protein, 23 aa, Escherichia coli
MLLKRRLIIAASLFVFNLSSGFA

SEQ ID NO: 82 - protein, 27 aa, Escherichia coli
MKKQTQLLSALALSVGLTLSASFQAVA

SEQ ID NO: 83 - protein, 28 aa, Escherichia coli
MFVKLLRSVAIGLIVGAILLVAMPSLRS

SEQ ID NO: 84 - protein, 23 aa, Escherichia coli
MNKKVLTLSAVMASMLFGAAAHA

SEQ ID NO: 85 - protein, 16 aa, Escherichia coli
MASSALTLPFSRIAHA

SEQ ID NO: 86 - protein, 28 aa, Escherichia coli
MRISLKKSGMLKLGLSLVAMTVAASVQA

SEQ ID NO: 87 - protein, 20 aa, Escherichia coli
MKKGFMLFTLLAAFSGFAQA

SEQ ID NO: 88 - protein, 17 aa, Escherichia coli
MKRKVLLIPLIIFLAIA

SEQ ID NO: 89 - protein, 17 aa, Escherichia coli
MLKKILLLALLPAIAFA

SEQ ID NO: 90 - protein, 18 aa, Escherichia coli
MIKHVLLFFVFISFSVSA

SEQ ID NO: 91 - protein, 19 aa, Escherichia coli
MSSKKIIGAFVLMTGILSG

SEQ ID NO: 92 - protein, 20 aa, Escherichia coli
MAKVISFFISLFLISFPLYA

SEQ ID NO: 93 - protein, 23 aa, Escherichia coli
MSFKKIIKAFVIMAALVSVQAHA

SEQ ID NO: 94 - protein, 25 aa, Escherichia coli
MSHYKTGHKQPRFRYSVLARCVAWA

SEQ ID NO: 95 - protein, 20 aa, Escherichia coli
MKTILPAVLFAAFATTSAWA

SEQ ID NO: 96 - protein, 25 aa, Escherichia coli
MFFNTKHTTALCFVTCMAFSSSSIA

SEQ ID NO: 97 - protein, 18 aa, Escherichia coli
MKNITFIFFILLASPLYA

SEQ ID NO: 98 - protein, 18 aa, Escherichia coli
MKNITFIFFILLASPLYA

SEQ ID NO: 99 - protein, 21 aa, Escherichia coli
MNKVKFYVLFTALLSSLCAHG

SEQ ID NO: 100 - protein, 21 aa, Escherichia coli
MNKVKCYVLFTALLSSLYAHG

SEQ ID NO: 101 - protein, 21 aa, Escherichia coli
MRRVNILCSFALLFASHTSLA

SEQ ID NO: 102 - protein, 21 aa, Escherichia coli
MKFLPYIFLLCCGLWSTISFA

SEQ ID NO: 103 - protein, 18 aa, Escherichia coli
MKKTIGLILILASFGSHA

SEQ ID NO: 104 - protein, 18 aa, Escherichia coli
MKKTIGLILILASFGSHA

SEQ ID NO: 105 - protein, 21 aa, Escherichia coli
MLKKIIPAIVLIAGTSGVVNA

SEQ ID NO: 106 - protein, 21 aa, Escherichia coli
MLKKIISAIALIAGTSGVVNA

SEQ ID NO: 107 - protein, 27 aa, Escherichia coli
MVMSQKTLFTKSALAVAVALISTQAWS

SEQ ID NO: 108 - protein, 35 aa, Escherichia coli
MKKYVTTKSVQPVAFRLTTLSLVMSAVLGSASVIA

SEQ ID NO: 109 - protein, 34 aa, Escherichia coli; identical to SEQ ID NO: 64

SEQ ID NO: 110 - protein, 22 aa, Escherichia coli
MKKTMMAAALVLSALSIQSALA

SEQ ID NO: 111 - protein, 24 aa, Escherichia coli
MKITHHYKSLLSAIISVALFYSAA

SEQ ID NO: 112 - protein, 20 aa, Escherichia coli
MKKVTLFLFVVSLLPSTVLA

SEQ ID NO: 113 - protein, 26 aa, Escherichia coli
MLNIIHRLKSGMFPALFFLTSASVLA

SEQ ID NO: 114 - protein, 22 aa, Escherichia coli
MKKTLLAIILGGMAFATTNASA

SEQ ID NO: 115 - protein, 19 aa, Escherichia coli
MNKFISIIALCVFSSYANA

SEQ ID NO: 116 - protein, 22 aa, Escherichia coli
MKNKYNLLFFLFLLCYGDVALA

SEQ ID NO: 117 - protein, 21 aa, Escherichia coli
MKKLYKAITVICILMSNLQSA

SEQ ID NO: 118 - protein, 20 aa, Escherichia coli
MIKKVPVLLFFMASISITHA

SEQ ID NO: 119 - protein, 21 aa, Escherichia coli
MNKYPPLLTMLIIGIGSNAVA

SEQ ID NO: 120 - protein, 33 aa, Escherichia coli
MTPLRVFRKTTPLVNTIRLSLLPLAGLSFSAFA

SEQ ID NO: 121 - protein, 21 aa, Escherichia coli
MLAFIRFLFAGLLLVISHAFA

SEQ ID NO: 122 - protein, 22 aa, Escherichia coli
MNKKIHSLALLVNLGIYGVAQA

SEQ ID NO: 123 - protein, 26 aa, Escherichia coli
MRLAPLYRNALLLTGLLLSGIAAVQA

SEQ ID NO: 124 - protein, 33 aa, Escherichia coli
MARSKTAQPKHSLRKIAVVVATAVSGMSVYAQA

SEQ ID NO: 125 - protein, 20 aa, Escherichia coli
MSKRIALFPALLLALLVIVA

SEQ ID NO: 126 - protein, 30 aa, Escherichia coli
MSGLPLISRRRLLTAMALSPLLWQMNTAHA

SEQ ID NO: 127 - protein, 36 aa, Escherichia coli
MLSTQFNRDNQYQAITKPSLLAGCIALALLPSAAFA

SEQ ID NO: 128 - protein, 36 aa, Escherichia coli
MSNKNVNVRKSQEITFCLLAGILMFMAMMVAGRAEA

SEQ ID NO: 129 - protein, 37 aa, Escherichia coli
MSYLNLRLYQRNTQCLHIRKHRLAGFFVRLVVACAFA

SEQ ID NO: 130 - protein, 20 aa, Escherichia coli
MRNKPFYLLCAFLWLAVSHA

SEQ ID NO: 131 - protein, 23 aa, Escherichia coli
MKWCKRGYVLAAILALASATIQA

SEQ ID NO: 132 - protein, 23 aa, Escherichia coli
MKRVITLFAVLLMGWSVNAWSFA

SEQ ID NO: 133 - protein, 25 aa, Escherichia coli
MKSLFKVTLLATTMAVALHAPITFA

SEQ ID NO: 134 - protein, 21 aa, Escherichia coli
MLIIKRSVAIIAILFSPLSTA

SEQ ID NO: 135 - protein, 21 aa, Escherichia coli
MQKNAAHTYAISSLLVLSLTG

SEQ ID NO: 136 - protein, 19 aa, Escherichia coli
MIKFLSALILLLVTTAAQA

SEQ ID NO: 137 - protein, 16 aa, Escherichia coli
MRTLLAILLFPLLVQA

SEQ ID NO: 138 - protein, 29 aa, Escherichia coli
MKLAHLGRQALMGVMAVALVAGMSVKSFA

SEQ ID NO: 139 - protein, 23 aa, Escherichia coli
MKIKTLAIVVLSALSLSSTAALA

SEQ ID NO: 140 - protein, 23 aa, Escherichia coli
MKLKFISMAVFSALTLGVATNAS

SEQ ID NO: 141 - protein, 23 aa, Escherichia coli
MRMKKSALTLAVLSSLFSGYSLA

SEQ ID NO: 142 - protein, 21 aa, Escherichia coli
MKKLAIIGATSVMMMTGTAQA

SEQ ID NO: 143 - protein, 21 aa, Escherichia coli
MKKLAIMAAASMVFAVSSAHA

SEQ ID NO: 144 - protein, 23 aa, Escherichia coli
MKFKKTIGAMALTTMFVAVSASA

SEQ ID NO: 145 - protein, 21 aa, Escherichia coli
MKKLAIMAAASMVFAVSSAHA

SEQ ID NO: 146 - protein, 21 aa, Escherichia coli
MIKSVIAGAVAMAVVSFGAYA

SEQ ID NO: 147 - protein, 21 aa, Escherichia coli
MQKIQFILGILAAASSSSTLA

SEQ ID NO: 148 - protein, 22 aa, Escherichia coli
MKKTLIALAVAASAAVSGSVMA

SEQ ID NO: 149 - protein, 21 aa, Escherichia coli
MKKLAIMAAASMIFTVGSAQA

SEQ ID NO: 150 - protein, 21 aa, Escherichia coli
MKRLVFISFVALSMTAGSAMA

SEQ ID NO: 151 - protein, 21 aa, Escherichia coli
MIKSVIAGAVAMAVVSFGANA

SEQ ID NO: 152 - protein, 21 aa, Escherichia coli
MKKAFLLACVFFLTGGGVSHA

SEQ ID NO: 153 - protein, 21 aa, Escherichia coli
MKKTLIALAIAASAASGMAHA

SEQ ID NO: 154 - protein, 23 aa, Escherichia coli
MKLKKTIGAMALATLFATMGASA

SEQ ID NO: 155 - protein, 22 aa, Escherichia coli
MLKIKYLLIGLSLSAMSSYSLA

SEQ ID NO: 156 - protein, 22 aa, Escherichia coli
MKKNLLITSVLAMATVSGSVLA

SEQ ID NO: 157 - protein, 21 aa, Escherichia coli
MRIWAVLASFLVFFYIPQSYA

SEQ ID NO: 158 - protein, 38 aa, Escherichia coli
MFFGDGGQLLSDKSLTGSAGGGNNRMKFNILPLAFFIG

SEQ ID NO: 159 - protein, 25 aa, Escherichia coli
MIKPTFLRRVAIAALLSGSCFSAAA

SEQ ID NO: 160 - protein, 22 aa, Escherichia coli
MKSVLKVSLAALTLAFAVSSHA

SEQ ID NO: 161 - protein, 25 aa, Escherichia coli
MKLTLKNLSMAIMMSTIVMGSSAMA

SEQ ID NO: 162 - protein, 25 aa, Escherichia coli
MFFKKNLTTAAICAALSVAAFSAMA

SEQ ID NO: 163 - protein, 27 aa, Escherichia coli
MDCVMKGLNKITCCLLAALLMPCAGHA

SEQ ID NO: 164 - protein, 21 aa, Escherichia coli
MKKVLGVILGGLLLLPVVSNA

SEQ ID NO: 165 - protein, 33 aa, Escherichia coli
MGYKMNISSLRKAFIFMGAVAALSLVNAQSALA

SEQ ID NO: 166 - protein, 22 aa, Escherichia coli
MKKLVLSLSLVLAFSSATAAFA

SEQ ID NO: 167 - protein, 20 aa, Escherichia coli
MKKWLLAAGLGLALATSAQA

SEQ ID NO: 168 - protein, 18 aa, Escherichia coli
MKQWIAALLLMLIPGVQA

SEQ ID NO: 169 - protein, 19 aa, Escherichia coli
MKKSILFIFLSVLSFSPFA

SEQ ID NO: 170 - protein, 19 aa, Escherichia coli
MKKSILFIFLSVLSFSPFP

SEQ ID NO: 171 - protein, 19 aa, Escherichia coli
MKKLMLAIFISVLSFPSFS

SEQ ID NO: 172 - protein, 26 aa, Escherichia coli
MKKTTLALSRLALSLGLALSPLSATA

SEQ ID NO: 173 - protein, 29 aa, Escherichia coli
MTIEYTKNYHHLTRIATFCALLYCNTAFS

SEQ ID NO: 174 - protein, 24 aa, Escherichia coli
MFSALRHRTAALALGVCFILPVHA

SEQ ID NO: 175 - protein, 25 aa, Escherichia coli
MMISKKYTLWALNPLLLTMMAPAVA

SEQ ID NO: 176 - protein, 20 aa, Escherichia coli
MKLFKSILLIAACHAAQASA

SEQ ID NO: 177 - protein, 23 aa, Escherichia coli
MNTKGKALLAGLIALAFSNMALA

SEQ ID NO: 178 - protein, 22 aa, Escherichia coli
MMKKIAITCALLSSLVASSVWA

SEQ ID NO: 179 - protein, 21 aa, Escherichia coli
MKKAKAIFLFILIVSGFLLVA

SEQ ID NO: 180 - protein, 19 aa, Escherichia coli
MKKITGIILLLLAVIILSA

SEQ ID NO: 181 - protein, 17 aa, Escherichia coli
MRKRFFVGIFAINLLVG

SEQ ID NO: 182 - protein, 17 aa, Escherichia coli
MCGKILLILFFIMTLSA

SEQ ID NO: 183 - protein, 19 aa, Escherichia coli
MKKITWIILLLLAAIILAA

SEQ ID NO: 184 - protein, 19 aa, Escherichia coli
MKKITGIILLLLAVIILAA

SEQ ID NO: 185 - protein, 19 aa, Escherichia coli
MKKITGIILLLLAAIILAA

SEQ ID NO: 186 - protein, 22 aa, Escherichia coli
MKMNKSLIVLCLSAGLLASAPG

SEQ ID NO: 187 - protein, 45 aa, Escherichia coli
MNNEETFYQAMRRQGVTRRSFLKYCSLAATSLGLGAGMAPKIAWA

SEQ ID NO: 188 - protein, 26 aa, Escherichia coli
MNRRNFIKAASCGALLTGALPSVSHA

SEQ ID NO: 189 - protein, 22 aa, Escherichia coli
MMKMRWLSAAVMLTLYTSSSWA

SEQ ID NO: 190 - protein, 19 aa, Escherichia coli
MNKTAIALLALLASSASLA

SEQ ID NO: 191 - protein, 20 aa, Escherichia coli
MKGRWVKYLLMGTVVAMLAA

SEQ ID NO: 192 - protein, 18 aa, Escherichia coli
MFKRRYVTLLPLFVLLAA

SEQ ID NO: 193 - protein, 16 aa, Escherichia coli
MKKYLALALIAPLLIS

SEQ ID NO: 194 - protein, 15 aa, Escherichia coli
MKAKAILLASVLLVG

SEQ ID NO: 195 - protein, 24 aa, Escherichia coli
MARKWLNLFAGAALSFAVAGNALA

SEQ ID NO: 196 - protein, 31 aa, Escherichia coli
MKLSRRSFMKANAVAAAAAAAGLSVPGVARA

SEQ ID NO: 197 - protein, 34 aa, Escherichia coli
MEFGSEIMKSHDLKKALCQWTAMLALVVSGAVWA

SEQ ID NO: 198 - protein, 29 aa, Escherichia coli
MKMRAVAVFTGMLTGVLSVTGLLSAGAYA

SEQ ID NO: 199 - protein, 27 aa, Escherichia coli
MDWLLDVFATWLYGLKVIAITLAVIMF

SEQ ID NO: 200 - protein, 22 aa, Escherichia coli
MLSTLRRTLFALLACASFIVHA

SEQ ID NO: 201 - protein, 23 aa, Escherichia coli
MKLTTHHLRTGAALLLAGILLAG

SEQ ID NO: 202 - protein, 24 aa, Escherichia coli
MAYSVQKSRLAKVAGVSLVLLLAA

SEQ ID NO: 203 - protein, 15 aa, Escherichia coli
MRFCLILITALLLAG

SEQ ID NO: 204 - protein, 25 aa, Escherichia coli
MSAGSPKFTVRRIAALSLVSLWLAG

SEQ ID NO: 205 - protein, 23 aa, Escherichia coli
MKKLTVAISAVAASVLMAMSAQA

SEQ ID NO: 206 - protein, 26 aa, Escherichia coli
MTRIKINARRIFSLLIPFFFFTSVHA

SEQ ID NO: 207 - protein, 32 aa, Escherichia coli
MSVLRSLLTAGVLASGLLWSLNGITATPAAQA

SEQ ID NO: 208 - protein, 27 aa, Escherichia coli
MNKGLLTLLLLFTCFAHAQVVDTWQFA

SEQ ID NO: 209 - protein, 21 aa, Escherichia coli
MKVKVLSLLVPALLVAGAANA

SEQ ID NO: 210 - protein, 22 aa, Escherichia coli
MMKRNILAVIVPALLVAGTANA

SEQ ID NO: 211 - protein, 17 aa, Escherichia coli
MKKLLPCTALVMCAGMA

SEQ ID NO: 212 - protein, 23 aa, Escherichia coli
MQTKLLAIMLAAPVVFSSQEASA

SEQ ID NO: 213 - protein, 20 aa, Escherichia coli
MRAKLLGIVLTTPIAISSFA

SEQ ID NO: 214 - protein, 23 aa, Escherichia coli
MKKIACLSALAAVLAFTAGTSVA

SEQ ID NO: 215 - protein, 26 aa, Escherichia coli
MTNITKRSLVAAGVLAALMAGNVALA

SEQ ID NO: 216 - protein, 23 aa, Escherichia coli
MFVTSKKMTAAVLAITLAMSLSA

SEQ ID NO: 217 - protein, 20 aa, Escherichia coli
MNKNMAGILSAAAVLTMLAG

SEQ ID NO: 218 - protein, 28 aa, Escherichia coli
MTMTRLKISKTLLAVMLTSAVATGSAYA

SEQ ID NO: 219 - protein, 24 aa, Escherichia coli
MKKRIPTLLATMIATALYSQQGLA

SEQ ID NO: 220 - protein, 20 aa, Escherichia coli
MRTLQGWLLPVFMLPMAVYA

SEQ ID NO: 221 - protein, 21 aa, Escherichia coli
MQLNKVLKGLMIALPVMAIAA

SEQ ID NO: 222 - protein, 22 aa, Escherichia coli
MIKSVIAGAVAMAVVSFGVNNA

SEQ ID NO: 223 - protein, 24 aa, Escherichia coli
MKDRIPFAVNNITCVILLSLFCNA

SEQ ID NO: 224 - protein, 21 aa, Escherichia coli
MIRKKILMAAIPLFVISGADA

SEQ ID NO: 225 - protein, 24 aa, Escherichia coli
MKKIRGLCLPVMLGAVLMSQHVHA

SEQ ID NO: 226 - protein, 18 aa, Escherichia coli
MIRLSLFISLLLTSVAVL

SEQ ID NO: 227 - protein, 21 aa, Escherichia coli
MKKWFPAFLFLSLSGGNDALA

SEQ ID NO: 228 - protein, 22 aa, Escherichia coli
MRLRFSVPLFFFGCVFVHGVFA

SEQ ID NO: 229 - protein, 27 aa, Escherichia coli
MVVNKTTAVLYLIALSLSGFIHTFLRA

SEQ ID NO: 230 - protein, 21 aa, Escherichia coli
MIKSTGALLLFAALSAGQAIA

SEQ ID NO: 231 - protein, 20 aa, Escherichia coli
MRFSRFIIGLTSCIAFSVQA

SEQ ID NO: 232 - protein, 28 aa, Escherichia coli
MLIMPKFRVSLFSLALMLAVPFAPQAVA

SEQ ID NO: 233 - protein, 27 aa, Escherichia coli
MPRLLTKRGCWITLAAAPFLLFLAAWG

SEQ ID NO: 234 - protein, 26 aa, Escherichia coli
MNAKIIASLAFTSMFSLSTLLSPAHA

SEQ ID NO: 235 - protein, 23 aa, Escherichia coli
MKKWSRHLLAAGALALGMSAAHA

SEQ ID NO: 236 - protein, 26 aa, Escherichia coli
MTALNKKWLSGLVAGALMAVSVGTLA

SEQ ID NO: 237 - protein, 22 aa, Escherichia coli
MKAILIPFLSLLIPLTPQSAFA

SEQ ID NO: 238 - protein, 22 aa, Escherichia coli
MNMFFRLTALAGLLAIAGQTFA

SEQ ID NO: 239 - protein, 21 aa, Escherichia coli
MRHSVLFATAFATLISTQTFA

SEQ ID NO: 240 - protein, 24 aa, Escherichia coli
MKKIRGLCLPVMLGAVLMSQHVHA

SEQ ID NO: 241 - protein, 18 aa, Escherichia coli
MIRLSLFISLLLTSVAVL

SEQ ID NO: 242 - protein, 21 aa, Escherichia coli
MKKWLPAFLFLSLSGCNDALA

SEQ ID NO: 243 - protein, 22 aa, Escherichia coli
MRLRFSVPLFFFGCVFVHGVFA

SEQ ID NO: 244 - protein, 27 aa, Escherichia coli
MVVNKTTAVLYLIALSLSGFIHTFLRA

SEQ ID NO: 245 - protein, 21 aa, Escherichia coli
MIKSTGALLLFAALSAGQAMA

SEQ ID NO: 246 - protein, 26 aa, Escherichia coli
MKRDGAMKITLLVTLLFGLVFLTTVG

SEQ ID NO: 247 - protein, 19 aa, Escherichia coli
MFKKGLLALALVFSLPVFA

SEQ ID NO: 248 - protein, 25 aa, Escherichia coli
MKVMRTTVATVVAATLSMSAFSVFA

SEQ ID NO: 249 - protein, 23 aa, Escherichia coli
MPRSTWFKALLLLVALWAPLSQA

SEQ ID NO: 250 - protein, 25 aa, Escherichia coli
MNMKKLATLVSAVALSATVSANAMA

SEQ ID NO: 251 - protein, 26 aa, Escherichia coli
MSGKPAARQGDMTQYGGSIVQGSAGV

SEQ ID NO: 252 - protein, 26 aa, Escherichia coli
MSGKPAARQGDMTQYGGPIVQGSAGV

SEQ ID NO: 253 - protein, 17 aa, Escherichia coli
MRKQWLGICIAAGMLAA

SEQ ID NO: 254 - protein, 18 aa, Escherichia coli
MRYLATLLLSLAVLITAG

SEQ ID NO: 255 - protein, 23 aa, Escherichia coli
MKAFWRNAALLAVSLLPFSSANA

SEQ ID NO: 256 - protein, 23 aa, Escherichia coli
MKQLWFAMSLVTGSLLFSANASA

SEQ ID NO: 257 - protein, 21 aa, Escherichia coli
MRQVLSSLLVIAGLVSGQAIA

SEQ ID NO: 258 - protein, 24 aa, Escherichia coli
MKLKFISMAVFSALTLGVATNASA

SEQ ID NO: 259 - protein, 27 aa, Escherichia coli
MVKDIIKTVTFSCMLAGSMFVTCHVCA

SEQ ID NO: 260 - protein, 23 aa, Escherichia coli
MAYSQPSFALLCRNNQTGQEFNS

SEQ ID NO: 261 - protein, 22 aa, Escherichia coli
MKLKAIILATGLINCIAFSAQA

SEQ ID NO: 262 - protein, 22 aa, Escherichia coli
MESINEIEGIYMKLRFISSALA

SEQ ID NO: 263 - protein, 23 aa, Escherichia coli
MMTKIKLLMLIIFYLIISASAHA

SEQ ID NO: 264 - protein, 20 aa, Escherichia coli
MRRVLFSCFCGLLWSSSGWA

SEQ ID NO: 265 - protein, 18 aa, Escherichia coli
MNMTKGALILSLSFLLAA

SEQ ID NO: 266 - protein, 27 aa, Escherichia coli
MEKAKQVTWRLLAAGVCLLTVSSVARA

SEQ ID NO: 267 - protein, 17 aa, Escherichia coli
MIKRVLVVSMVGLSLVG

SEQ ID NO: 268 - protein, 30 aa, Escherichia coli
MARTKLKFRLHRAVIVLFCLALLVALMQGA

SEQ ID NO: 269 - protein, 19 aa, Escherichia coli
MKRFSLAILALVVATGAQA

SEQ ID NO: 270 - protein, 26 aa, Escherichia coli
MVKSQPILRYILRGIPAIAVAVLLSA

SEQ ID NO: 271 - protein, 23 aa, Escherichia coli
MRKLTALFVASTLALGAANLAHA

SEQ ID NO: 272 - protein, 19 aa, Escherichia coli
MNKWGVGLTFLLAATSVMA

SEQ ID NO: 273 - protein, 27 aa, Escherichia coli
MSLSRRQFIQASGIALCAGAVPLKASA

SEQ ID NO: 274 - protein, 20 aa, Escherichia coli
MKNWKTLLLGIAMIANTSFA

SEQ ID NO: 275 - protein, 22 aa, Escherichia coli
MAISSRNTLLAALAFIAFQAQA

SEQ ID NO: 276 - protein, 18 aa, Escherichia coli
MLKKCLPLLLLCTAPVFA

SEQ ID NO: 277 - protein, 26 aa, Escherichia coli
MMNFNNVFRWHLPFLFLVLLTFRAAA

SEQ ID NO: 278 - protein, 21 aa, Escherichia coli
MKQALRVAFGFLILWASVLHA

SEQ ID NO: 279 - protein, 24 aa, Escherichia coli
MQMKKLLPILIGLSLSGFSSLSQA

SEQ ID NO: 280 - protein, 39 aa, Escherichia coli
MNNNDLFQASRRRFLAQLGGLTVAGMLGPSLLTPRRATA

SEQ ID NO: 281 - protein, 19 aa, Escherichia coli
MNKALLPLLLCCFIFPASG

SEQ ID NO: 282 - protein, 18 aa, Escherichia coli
MKRILPLILALVAGMAQA

SEQ ID NO: 283 - protein, 22 aa, Escherichia coli
MKRRLWLLMLFLFAGHVPAASA

SEQ ID NO: 284 - protein, 18 aa, Escherichia coli
MKQTSFFIPLLGTLLLYG

SEQ ID NO: 285 - protein, 18 aa, Escherichia coli
MRCRGLIALLIWGQSVAA

SEQ ID NO: 286 - protein, 22 aa, Escherichia coli
MSLTKSLLFTLLLSAAAVQAST

SEQ ID NO: 287 - protein, 21 aa, Escherichia coli
MKLSMKSLAALLMMLNGAVMA

SEQ ID NO: 288 - protein, 30 aa, Escherichia coli
MKSPAPSRPQKMALIPACIFLCFAALSVQA

SEQ ID NO: 289 - protein, 30 aa, Escherichia coli
MSRFQRLTKYVAIGGGAALLLAGAAYLAGA

SEQ ID NO: 290 - protein, 30 aa, Escherichia coli
MSRILKRIAAGVVIAGVAALLLAAGGYAAG

SEQ ID NO: 291 - protein, 24 aa, Escherichia coli
MMPRIKPLLVLCAALLTVTPAASA

SEQ ID NO: 292 - protein, 20 aa, Escherichia coli
MKMKKLMMVALVSSTLALSG

SEQ ID NO: 293 - protein, 21 aa, Escherichia coli
MMKTKKLMMVALVSSTLALSG

SEQ ID NO: 294 - protein, 22 aa, Escherichia coli
MKHNVKLMAMTAVLSSVLVLSG

SEQ ID NO: 295 - protein, 20 aa, Escherichia coli
MKTKKLMMVALVSSTLALSG

SEQ ID NO: 296 - protein, 22 aa, Escherichia coli
MKKTLLAAGAVLALSSSFTVNA

SEQ ID NO: 297 - protein, 23 aa, Escherichia coli
MKPLHYTASALALGLALMGNAQA

SEQ ID NO: 298 - protein, 19 aa, Escherichia coli
MAMAVICLTAASGLTSAYA

SEQ ID NO: 299 - protein, 20 aa, Escherichia coli
MAMKKLLIASLLFSSATVYG

SEQ ID NO: 300 - protein, 28 aa, Escherichia coli
MGRISSGGMMFKAITTVAALVIATSAMA

SEQ ID NO: 301 - protein, 25 aa, Escherichia coli
MKLLQRGVALALLTTFTLASETALA

SEQ ID NO: 302 - protein, 17 aa, Escherichia coli
MKLRLSALALGTTLLVG

SEQ ID NO: 303 - protein, 16 aa, Escherichia coli
MKKMLLATALALLITG

SEQ ID NO: 304 - protein, 20 aa, Escherichia coli
MMKSKMKLMPLLVSVTLISG

SEQ ID NO: 305 - protein, 23 aa, Escherichia coli
MKIKNILLTLCTSLLLTNVAAHA

SEQ ID NO: 306 - protein, 23 aa, Escherichia coli
MKSVFTISASLAISLMLCCTAQA

SEQ ID NO: 307 - protein, 20 aa, Escherichia coli
MKTFFRTVLFGSLMAVCANS

SEQ ID NO: 308 - protein, 28 aa, Escherichia coli
MQRRDFLKYSVALGVASALPLWSRAVFA

SEQ ID NO: 309 - protein, 21 aa, Escherichia coli
MKTIFRYILFLALYSCCNTVS

SEQ ID NO: 310 - protein, 19 aa, Escherichia coli
MYKQAVILLLMLFTASVSA

SEQ ID NO: 311 - protein, 28 aa, Escherichia coli
MMTFKNLRYGLSSSVVLAASLFSVLSYA

SEQ ID NO: 312 - protein, 23 aa, Escherichia coli
MIKTTPHKIVILMGILLSPSVFA

SEQ ID NO: 313 - protein, 23 aa, Escherichia coli
MSKKLGFALSGLMLAMVAGTASA

SEQ ID NO: 314 - protein, 22 aa, Escherichia coli
MAKSLFRALVALSFLAPLWLNA

SEQ ID NO: 315 - protein, 22 aa, Escherichia coli
MAFKFKTFAAVGALIGSLALVG

SEQ ID NO: 316 - protein, 38 aa, Escherichia coli
MPERVLNFRALFLHGLYKMDKPKAYCRLFLPSFLLLSA

SEQ ID NO: 317 - protein, 23 aa, Escherichia coli
MSLNFSAFSDVLSPLAECAPTFA

SEQ ID NO: 318 - protein, 19 aa, Escherichia coli
MRKIALILAMLLIPCVSFA

SEQ ID NO: 319 - protein, 17 aa, Escherichia coli
MSLPSIPSFVLSGLLLI

SEQ ID NO: 320 - protein, 18 aa, Escherichia coli
MNSKKLCCICVLFSLLAG

SEQ ID NO: 321 - protein, 29 aa, Escherichia coli
MPLRRFSPGLKAQFAFGMVFLFVQPDASA

SEQ ID NO: 322 - protein, 18 aa, Escherichia coli
MKKHLLPLALLFSGISPA

SEQ ID NO: 323 - protein, 22 aa, Escherichia coli
MKKKVLAIALVTVFTGMGVAQA

SEQ ID NO: 324 - protein, 21 aa, Escherichia coli
MKIISKMLVGALALAVTNVYA

SEQ ID NO: 325 - protein, 27 aa, Escherichia coli
MHSWKKKLVVSQLALACTLAITSQANA

SEQ ID NO: 326 - protein, 17 aa, Escherichia coli
MFKKILFPLVALFMLAG

SEQ ID NO: 327 - protein, 18 aa, Escherichia coli
MKLVHMASGLAVAIALAA

SEQ ID NO: 328 - protein, 17 aa, Escherichia coli
MKYSSIFSMLSFFILFA

SEQ ID NO: 329 - protein, 20 aa, Escherichia coli
MRKFIFVLLTLLLVSPFSFA

SEQ ID NO: 330 - protein, 28 aa, Escherichia coli
MNKEQSADDPSVDLIRVKNMLNSTISMS

SEQ ID NO: 331 - protein, 15 aa, Escherichia coli
MDLNEASLNAASTRA

SEQ ID NO: 332 - protein, 22 aa, Escherichia coli
MQLRKPATAILALALSAGLAQA

SEQ ID NO: 333 - protein, 21 aa, Escherichia coli
MELYREYPAWLIFLRRTYAVA

SEQ ID NO: 334 - protein, 19 aa, Escherichia coli
MKRVLFFLLMIFVSFGVIA

SEQ ID NO: 335 - protein, 16 aa, Escherichia coli
MKKLILIAIMASGLVA

SEQ ID NO: 336 - protein, 22 aa, Escherichia coli
MKTNRSLVVIVSLITATLLLTA

SEQ ID NO: 337 - protein, 23 aa, Escherichia coli
MFKGQKTLAALAVSLLFTAPVYA

SEQ ID NO: 338 - protein, 26 aa, Escherichia coli
MSSNFRHQLLSLSLLVGIAAPWAAFA

SEQ ID NO: 339 - protein, 32 aa, Escherichia coli
MPFTSSGGEMSAGKGLLLVICLLFLPLKSAMA

SEQ ID NO: 340 - protein, 24 aa, Escherichia coli
MDTVNIYRLSFVSCLVMAMPCAMA

SEQ ID NO: 341 - protein, 21 aa, Escherichia coli
MNTFSVSRLALALAFGVTLTA

SEQ ID NO: 342 - protein, 18 aa, Escherichia coli
MGKAVIAIHGGAGAISRA

SEQ ID NO: 343 - protein, 22 aa, Escherichia coli
MNMKLKTLFAAAFAVVGFCSTA

SEQ ID NO: 344 - protein, 22 aa, Escherichia coli
MIMKNCLLLGALLMGFTGVAMA

SEQ ID NO: 345 - protein, 18 aa, Escherichia coli
MRYSKLTMLIPCALLLSA

SEQ ID NO: 346 - protein, 35 aa, Escherichia coli
MPARHLYFIMTNTWNRLALLIFAVLSLLVAGELQA

SEQ ID NO: 347 - protein, 24 aa, Escherichia coli
MITMKKSVLTAFITVVCATSSVMA

SEQ ID NO: 348 - protein, 26 aa, Escherichia coli
MKTCITKGIVTVSLTAILLSCSSAWA

SEQ ID NO: 349 - protein, 30 aa, Escherichia coli
MYRTHRQHSLLSSGGVPSFIGGLVVFVSAA

SEQ ID NO: 350 - protein, 20 aa, Escherichia coli
MKKNIFKFSVLTLAVLSLTA

SEQ ID NO: 351 - protein, 35 aa, Escherichia coli
MQYKDENGVNEPSRRRLLKVIGALALAGSCPVAHA

SEQ ID NO: 352 - protein, 20 aa, Escherichia coli
MLRNGNKYLLMLVSIIMLTA

SEQ ID NO: 353 - protein, 26 aa, Escherichia coli
MYSSSRKRCPKTKWALKLLTAAFLAA

SEQ ID NO: 354 - protein, 16 aa, Escherichia coli
MYPVDLHMHTVASTHA

SEQ ID NO: 355 - protein, 22 aa, Escherichia coli
MKKSLLGLTFASLMFSAGSAVA

SEQ ID NO: 356 - protein, 23 aa, Escherichia coli
MMIKTRFSRWLTFFTFAAAVALA

SEQ ID NO: 357 - protein, 22 aa, Escherichia coli
MNMMRIFYIGLSGVGMMFSSMA

SEQ ID NO: 358 - protein, 22 aa, Escherichia coli
MKIKSIRKAVLLLALLTSTSFA

SEQ ID NO: 359 - protein, 25 aa, Escherichia coli
MSTTHNVPQGDLVLRTLAMPADTNA

SEQ ID NO: 360 - protein, 21 aa, Escherichia coli
MKKLTVAALAVTTLLSGSAFA

SEQ ID NO: 361 - protein, 16 aa, Escherichia coli
MLELLFLLLPVAAAYG

SEQ ID NO: 362 - protein, 16 aa, Escherichia coli
MKILYSFLLLPFFSCA

SEQ ID NO: 363 - protein, 22 aa, Escherichia coli
MKKENYSFKQACAVVGGQSAMA

SEQ ID NO: 364 - protein, 23 aa, Escherichia coli
MMMKKTLLLCAFLVGLVSSNVMA

SEQ ID NO: 365 - protein, 20 aa, Escherichia coli
MKKLALILFMGTLVSFYADA

SEQ ID NO: 366 - protein, 22 aa, Escherichia coli
MAMAAVCGTSGIASLFSQAAFA

SEQ ID NO: 367 - protein, 20 aa, Escherichia coli
MTVSSHRLELLSPARDAAIA

SEQ ID NO: 368 - protein, 19 aa, Escherichia coli
MKFQAIVLASFLVMPYALA

SEQ ID NO: 369 - protein, 18 aa, Escherichia coli
MNRIYRVIWNCTLQVFQA

SEQ ID NO: 370 - protein, 27 aa, Escherichia coli
MARINRISITLCALLFTTLPLTPMAHA

SEQ ID NO: 371 - protein, 22 aa, Escherichia coli
MTKLKLLALGVLIATSAGVAHA

SEQ ID NO: 372 - protein, 19 aa, Escherichia coli
MEKNMKKRGAFLGLLLVSA

SEQ ID NO: 373 - protein, 20 aa, Escherichia coli
MEIRIMLFILMMMVMPVSYA

SEQ ID NO: 374 - protein, 22 aa, Escherichia coli
MLRMTPLASAIVALLLGIEAYA

SEQ ID NO: 375 - protein, 31 aa, Escherichia coli
MAAIPWRPFNLRGIKMKGLLSLLIFSMVLPA

SEQ ID NO: 376 - protein, 22 aa, Escherichia coli
MKRSIIAAAVFSSFFMSAGVFA

SEQ ID NO: 377 - protein, 22 aa, Escherichia coli
MNKYWLSGIIFLAYGLASPAFS

SEQ ID NO: 378 - protein, 26 aa, Escherichia coli
MEFYMKAFNKLFSLVVASVLVFSLAG

SEQ ID NO: 379 - protein, 25 aa, Escherichia coli
MYDFNLVLLLLQQMCVFLVIAWLMS

SEQ ID NO: 380 - protein, 23 aa, Escherichia coli
MPLLKLWAGSLVMLAAVSLPLQA

SEQ ID NO: 381 - protein, 21 aa, Escherichia coli
MQMIVRILLLFIALFTFGVQA

SEQ ID NO: 382 - protein, 23 aa, Escherichia coli
MRIIFLRKEYLSLLPSMIASLFS

SEQ ID NO: 383 - protein, 28 aa, Escherichia coli
MSDLLCSAKLGAMTLALLLSATSLSALA

SEQ ID NO: 384 - protein, 29 aa, Escherichia coli
MPDHSLFRLRILPWCIALAMSGSYSSVWA

SEQ ID NO: 385 - protein, 19 aa, Escherichia coli
MRKYIPLVLFIFSWPVLCA

SEQ ID NO: 386 - protein, 18 aa, Escherichia coli
MIKHLVAPLVFTSLILTG

SEQ ID NO: 387 - protein, 23 aa, Escherichia coli
MKIRSLSRFVLASTMFASFTASA

SEQ ID NO: 388 - protein, 23 aa, Escherichia coli
MSAFKKSLLVAGVAMILSNNVFA

SEQ ID NO: 389 - protein, 22 aa, Escherichia coli
MPKLRLIGLTLLALSATAVSHA

SEQ ID NO: 390 - protein, 20 aa, Escherichia coli
MKKFAAVIAVMALCSAPVMA

SEQ ID NO: 391 - protein, 21 aa, Escherichia coli
MKLITAPCRALLALPFCYAFS

SEQ ID NO: 392 - protein, 22 aa, Escherichia coli
MKIKTILTPVTCALLISFSAHA

SEQ ID NO: 393 - protein, 27 aa, Escherichia coli
MKFKTNKLSLNLVLASSLLAASIPAFA

SEQ ID NO: 394 - protein, 15 aa, Escherichia coli
MELLCPAGNLPALKA

SEQ ID NO: 395 - protein, 21 aa, Escherichia coli
MLKKTLLAYTIGFAFSPPANA

SEQ ID NO: 396 - protein, 20 aa, Escherichia coli
MNNVKLLIAGSAFFAMSAQA

SEQ ID NO: 397 - protein, 19 aa, Escherichia coli
MRRLPGILLLTGAALVVIA

SEQ ID NO: 398 - protein, 22 aa, Escherichia coli
MQCGISDGLPGFSYADADGKFS

SEQ ID NO: 399 - protein, 24 aa, Escherichia coli
MKLNIFTKSMIGMGLVCSALPALA

SEQ ID NO: 400 - protein, 17 aa, Escherichia coli
MKRLLILTALLPFVGFA

SEQ ID NO: 401 - protein, 19 aa, Escherichia coli
MDKSKRHLAWWVVGLLAVA

SEQ ID NO: 402 - protein, 20 aa, Escherichia coli
MNRRRKLLIPLLFCGAMLTA

SEQ ID NO: 403 - protein, 24 aa, Escherichia coli
MQGTKIRLLAGGLLMMATAGYVQA

SEQ ID NO: 404 - protein, 21 aa, Escherichia coli
MNVLRSGIVTMLLLAAFSVQA

SEQ ID NO: 405 - protein, 25 aa, Escherichia coli
MKRKLFWICAVAMGMSAFPSFMTQA

SEQ ID NO: 406 - protein, 20 aa, Escherichia coli
MKKRVYLIAAVVSGALAVSG

SEQ ID NO: 407 - protein, 24 aa, Escherichia coli
MKLRSVTYALFIAGLAAFSTSSLA

SEQ ID NO: 408 - protein, 21 aa, Escherichia coli
MLINRNIVALFALPFMASATA

SEQ ID NO: 409 - protein, 15 aa, Escherichia coli
MDLLIILTYVAFAWA

SEQ ID NO: 410 - protein, 19 aa, Escherichia coli
MKACLLLFFYFSFICQLHG

SEQ ID NO: 411 - protein, 15 aa, Escherichia coli
MLLHILYLVGITAEA

SEQ ID NO: 412 - protein, 26 aa, Escherichia coli
MMNVSEKMEHFDVAIIGLGPAGSALA

SEQ ID NO: 413 - protein, 23 aa, Escherichia coli
MDFSIMVYAVIALVGVAIGWLFA

SEQ ID NO: 414 - protein, 22 aa, Escherichia coli
MMRIVTAAVMASTLAVSSLSHA

SEQ ID NO: 415 - protein, 23 aa, Escherichia coli
MKPGCTLFFLLCSALTVTTEAHA

SEQ ID NO: 416 - protein, 18 aa, Escherichia coli
MKNRLLILSLLVSVPAFA

SEQ ID NO: 417 - protein, 18 aa, Escherichia coli
MKASLALLSLLTAFTSHS

SEQ ID NO: 418 - protein, 21 aa, Escherichia coli
MKKVLYGIFAISALAATSAWA

SEQ ID NO: 419 - protein, 25 aa, Escherichia coli
MVILPLWRRVVKRPALILICLLLQA

SEQ ID NO: 420 - protein, 20 aa, Escherichia coli
MIKQTIVALLLSVGASSVFA

SEQ ID NO: 421 - protein, 17 aa, Escherichia coli
MKKRHLLSLLALGISTA

SEQ ID NO: 422 - protein, 17 aa, Escherichia coli
MKKIIALMLFLTFFAHA

SEQ ID NO: 423 - protein, 23 aa, Escherichia coli
MNKYLKYFSGTLVGLMLSTSAFA

SEQ ID NO: 424 - protein, 24 aa, Escherichia coli
MMNRVIPLPDEQATLDLGERVAKA

SEQ ID NO: 425 - protein, 33 aa, Escherichia coli
MASSSLIMGNNMHVKYLAGIVGAALLMAGCSSS

SEQ ID NO: 426 - protein, 19 aa, Escherichia coli
MRLIITFLMAWCLSWGAYA

SEQ ID NO: 427 - protein, 18 aa, Escherichia coli
MGILKSLFTLGKSFISQA

SEQ ID NO: 428 - protein, 20 aa, Escherichia coli
MFSRVLALLAVLLLSANTWA

SEQ ID NO: 429 - protein, 26 aa, Escherichia coli
MKFMKKAKILSGVLLLCFSSPLISQA

SEQ ID NO: 430 - protein, 21 aa, Escherichia coli
MNAIISPDYYYVLTVAGQSNA

SEQ ID NO: 431 - protein, 26 aa, Escherichia coli
MMMKTVKHLLCCAIAASALISTGVHA

SEQ ID NO: 432 - protein, 23 aa, Escherichia coli
MTILSLSRFMLAGVLLASFNASA

SEQ ID NO: 433 - protein, 22 aa, Escherichia coli
MKKALQVAMFSLFTVIGFNAQA

SEQ ID NO: 434 - protein, 28 aa, Escherichia coli
MKKIALIIGSMIAGGIISAAGFTWVAKA

SEQ ID NO: 435 - protein, 20 aa, Escherichia coli
MHRQSFFLVPLICLSSALWA

SEQ ID NO: 436 - protein, 18 aa, Escherichia coli
MKKNSYLLSCLAIAVSSA

SEQ ID NO: 437 - protein, 22 aa, Escherichia coli
MNKLQSYFIASVLYVMTPHAFA

SEQ ID NO: 438 - protein, 15 aa, Escherichia coli
MRPLILSIFALFLAG

SEQ ID NO: 439 - protein, 23 aa, Escherichia coli
MKRASLLTLTLIGAFSAIQAAWA

SEQ ID NO: 440 - protein, 19 aa, Escherichia coli
MEISFTRVALLAAALFFVG

SEQ ID NO: 441 - protein, 26 aa, Escherichia coli
MPTKMRTTRNLLLMATLLGSALFARA

SEQ ID NO: 442 - protein, 18 aa, Escherichia coli
MKIILLFLAALASFTVHA

SEQ ID NO: 443 - protein, 19 aa, Escherichia coli
MKTIFTVGAVVLATCLLSG

SEQ ID NO: 444 - protein, 28 aa, Escherichia coli
MKLMRYLNTKNIIAAGVLLSCMSSIAWG

SEQ ID NO: 445 - protein, 21 aa, Escherichia coli
MRYLLIVITFFMGFSSLPAWA

SEQ ID NO: 446 - protein, 23 aa, Escherichia coli
MEGSRMKYRIALAVSLFALSAGS

SEQ ID NO: 447 - protein, 22 aa, Escherichia coli
MNKVTKTAIAGLLALFAGNAAA

SEQ ID NO: 448 - protein, 25 aa, Escherichia coli
MSKRTFAVILTLLCSFCIGQALAGG

SEQ ID NO: 449 - protein, 40 aa, Escherichia coli
MPQRHHQGHKRTPKQLALIIKRCLPMVLTGSGMLCTTANA

SEQ ID NO: 450 - protein, 20 aa, Escherichia coli
MKRAPLITGLLLISTSCAYA

SEQ ID NO: 451 - protein, 23 aa, Escherichia coli
MKALSPIAVLISALLLQGCVAAA

SEQ ID NO: 452 - protein, 21 aa, Escherichia coli
MFKRLMMVALLVIAPLSAATA

SEQ ID NO: 453 - protein, 28 aa, Escherichia coli
MQTKKNEIWVGIFLLAALLAALFVCLKA

SEQ ID NO: 454 - protein, 20 aa, Escherichia coli
MKKINAIILLSSLTSASVFA

SEQ ID NO: 455 - protein, 20 aa, Escherichia coli
MTLRKILALTCLLLPMMASA

SEQ ID NO: 456 - protein, 21 aa, Escherichia coli
MRYIRQLCCVSLLCLSGSAVA

SEQ ID NO: 457 - protein, 21 aa, Escherichia coli
MWKRLLIVSAVSAAMSSMALA

SEQ ID NO: 458 - protein, 26 aa, Escherichia coli
MRVIMKPLRRTLVFFIFSVFLCGTVS

SEQ ID NO: 459 - DNA, 78 bp, Escherichia coli
atgaaaataaaaacaggtgcacgcatcctcgcattatccgcattaacgacgatgatgttttccgcctcggctctcgcc

SEQ ID NO: 460 - DNA, 75 bp, Escherichia coli
atgatgattactctgcgcaaacttcctctggcggttgccgtcgcagcgggcgtaatgtctgctcaggcaatggct

SEQ ID NO: 461 - DNA, 66 bp, Escherichia coli
atgaaatacctattgcctacggcagccgctggattgttattactcgcggcccagccggccatggcc

SEQ ID NO: 462 - DNA, 69 bp, Escherichia coli
atgaaacggaatgcgaaaactatcatcgcagggatgattgcactggcaatttcacacaccgctatggct

SEQ ID NO: 463 - DNA, 54 bp, Escherichia coli
atgcgcgtactgctatttttacttctttcccttttcatgttgccggcattttcg

SEQ ID NO: 464 - DNA, 63 bp, Escherichia coli
atgaagcaggcattacgagtagcatttggttttctcatactgtgggcatcagttctgcatgct

SEQ ID NO: 465 - DNA, 57 bp, Escherichia coli
atgaaaaagatttggctggcgctggctggtttagttttagcgtttagcgcatcggcg

SEQ ID NO: 466 - DNA, 78 bp, Escherichia coli
atgaaaaatagaaatcgtatgatcgtgaactgtgttactgcttccctgatgtattattggagcttacctgcactggct

SEQ ID NO: 467 - DNA, 126 bp, Escherichia coli
atgaacaataacgatctctttcaggcatcacgtcggcgttttctggcacaactcggcggcttaaccgtcgccgggatgctggggccgtcattgttaacgccgcgacgtgcgactgcggcgcaagcg

SEQ ID NO: 468 - DNA, 63 bp, Escherichia coli
atgaaacaaagcactattgcactggcactcttaccgttgctcttcacccctgttaccaaagcc

SEQ ID NO: 469 - DNA, 63 bp, Escherichia coli
atgaaaaagacagctatcgcgattgcagtggcactggctggtttcgctaccgtagcgcaggcc

(19) United States
(12) Patent Application Publication
(10) Pub. No.: US 2010/0168392 A1
(43) Pub. Date: Jul. 1, 2010

(54) EXPRESSION OF FULL LENGTH IGG AND SECRETION INTO THE CULTURE MEDIUM OF PROKARYOTIC CELLS

(75) Inventors: Ralf Ostendorp, Munich (DE); Andreas Popp, Kaufering (DE); Martina Fischer, Munich (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 12/672,859

(22) PCT Filed: Aug. 10, 2007
(86) PCT No.: PCT/EP07/58333

§ 371 Date: Feb. 26, 2010

(51) Int. Cl. C07K 16/00; C12P 21/06

(52) U.S. Cl. 530/387.1; 435/69.6

(57) ABSTRACT

A method for the production of an immunoglobulin or a functional fragment thereof in a prokaryotic host cell comprises transforming the host cell with (a) a first nucleic acid molecule comprising a nucleic acid sequence encoding a V_{L} and a C_{L} region and (b) a second nucleic acid molecule comprising a nucleic acid sequence encoding a V_{H}, a C_{H1}, a C_{H2} and at least a portion of a C_{H3} region, The host cell is within culture medium. The host cell is cultured under conditions so as to allow the host cell (a) to express (1) the V_{L} and a C_{L} region and (2) the V_{H}, the C_{H1}, the C_{H2} and the portion of the C_{H3} region, and (b) to secrete (a)(1) and (a)(2) to the periplasm of the host cell and thereafter to the culture medium of the host cell. Characteristically, (a)(1) and (a)(2) interact to form the immunoglobulin or functional fragment thereof.

### BACKGROUND OF THE INVENTION

The expression of heterologous genes in bacteria is known in the art almost since modern recombinant DNA technology was available in the 1970's. Various strategies have been employed, mostly directed by the needs, the circumstances and the technological advances available at a certain time. The first recombinant Escherichia coli strain was generated by Cohen et al in 1973 (Proc Natl Acad Sci USA. 1973 November; 70(11):3240-3244). In 1977 the first human gene, somatostatin, was expressed in prokaryotes (Science, 1977, 198, 1056-1063). Villa-Komaroff et al. (Proc Natl Acad Sci USA, 1978, 75(B), 3727-3731) for the first time directed the expression of a heterologous gene, proinsulin, into the periplasm. The secretion of heterologous genes into the culture medium of prokaryotes was not accomplished until 1989 (WO91/06655, Schering Corporation). Such secretion however was limited to homomeric and relatively short proteins and polypeptides.

The production and secretion of more complex polypeptides, such as multimeric or heteromeric polypeptides was therefore another obstacle that had to be overcome. Only in 2002 Simmons et al. described the expression of full-length immunoglobulins in Escherichia coli and their secretion into the periplasm (J Immunol Methods. 2002 May 1; 263(1-2):133-47). In 2007 Mazor et al. describe the isolation of full-length IgG antibodies from combinatorial libraries expressed in E. coli (Nat. Biotechnol. 2007 May; 25(5):563-5. Epub 2007 Apr. 15). However, the method described by Mazor et al. still requires the permeabilization of the outer membrane to release the immunoglobulins into the culture medium.

### SUMMARY OF THE INVENTION

The present invention overcomes the long felt need to produce full-length immunoglobulins in prokaryotic cells, thereby secreting the immunoglobulins produced into the culture medium. This enables the easy and convenient purification of functional immunoglobulins directly from the cell culture medium of prokaryotic cells.

In one embodiments the invention describes a method for the production of an immunoglobulin or a functional fragment thereof in a prokaryotic host cell, said method comprising:
i) transforming said host cell with (a) a first nucleic acid molecule comprising a nucleic acid sequence encoding a V_{L} and a C_{L} region and (b) a second nucleic acid molecule comprising a nucleic acid sequence encoding a V_{H}, a C_{H1}, a C_{H2} and at least a portion of a C_{H3} region, wherein said host cell is comprised within culture medium;
ii) culturing said host cell under conditions so as to allow said host cell (a) to encode (1) said V_{L} and a C_{L} region and (2) said V_{H}, said C_{H1}, said C_{H2} and said portion of said C_{H3} region, and (b) to secrete (a)(1) and (a)(2) to the periplasm of said host cell and thereafter to the culture medium of said host cell, wherein (a)(1) and (a)(2) interact to form said immunoglobulin or functional fragment thereof.

In preferred embodiments of the invention, said light chain of the immunoglobulin comprises a V_{L} and a C_{L} region. In other preferred embodiments said heavy chain of the immunoglobulin comprises a V_{H}, a C_{H1}, a C_{H2} and at least a portion of a C_{H3} region. In alternative preferred embodiments said heavy chain of the immunoglobulin comprises comprises a V_{H}, a C_{H1}, a C_{H2} and a full-length C_{H3} region. In certain embodiments said immunoglobulin is a functional fragment of said immunoglobulin. In other preferred embodiments said immunoglobulin is a full-length immunoglobulin.

In preferred embodiments of the invention, said immunoglobulin is of the IgG type, most preferably of the IgG1 type.

In certain embodiments of the invention, the first nucleic acid molecule, which contains a nucleic acid sequence encoding a light chain of an immunoglobulin, further comprises a nucleic acid sequence encoding for a signal sequence. In other embodiments of the invention, the second nucleic acid molecule, which contains a nucleic acid sequence encoding a heavy chain of an immunoglobulin, further comprises a nucleic acid sequence encoding for a signal sequence. In most preferred embodiments, both the first nucleic acid molecule comprising a nucleic acid sequence encoding a light chain of an immunoglobulin and the second nucleic acid molecule comprising a nucleic acid sequence encoding a heavy chain of an immunoglobulin further comprise a nucleic acid sequence encoding for a signal sequence. In some embodiments these two signal sequences are identical. In other, preferred, embodiments, the two signal sequences are different. In certain particular embodiments, the signal sequence comprised in the second nucleic acid molecule comprising a nucleic acid sequence encoding a heavy chain of an immunoglobulin is the signal sequence of gene phoA of Escherichia coli.

In preferred embodiments of the invention the signal sequences is a prokaryotic signal sequence. Most preferred is a signal sequences of Escherichia coli, in particular of MalE, LamB, PelB, LivK, TorT, TolB, DsbA, Pac, TorA, PhoA and OmpA, more particularly LamB, PelB, LivK, TorT, TolB, DsbA, Pac, TorA, PhoA and OmpA, and most particularly LamB, PelB, LivK, DsbA, Pac and OmpA. In alternative embodiments, the signal sequences can be a eukaryotic signal sequence. In preferred embodiments, a signal sequence is, e.g., N-terminal with respect to the heavy chain and the light chain.

In certain embodiments of the invention, the method further comprises the steps of recovering said immunoglobulin or said functional fragment thereof from the culture medium. In yet further embodiments, the method further comprises the step of purifying said immunoglobulin or said functional fragment thereof.

In certain preferred embodiments of the invention, the first and the second nucleic acid molecules are operably linked to the same promoter. In alternative embodiments, the first and the second nucleic acid molecules are not operably linked to the same promoter.

In certain preferred embodiments of the invention, the first and second nucleic acid molecules are comprised within the same vector.

In another embodiment the invention relates to an immunoglobulin or a functional fragment thereof, produced according to the present invention. In preferred embodiments said immunoglobulin is a full-length immunoglobulin. In preferred embodiments said immunoglobulin or a functional fragment, produced according to the present invention is aglycosylated.

Yet other embodiments of the invention relate to the use of a prokaryotic host cell cell for the production of an immunoglobulin or a functional fragment thereof, wherein said immunoglobulin or said functional fragment thereof is secreted into the culture medium. In preferred embodiments said immunoglobulin or functional fragment thereof comprises a V_{L} and a C_{L} region and a V_{H}, a C_{H1}, a C_{H2} and at least a portion of a C_{H3} region.

In preferred embodiments, the prokaryotic host cell used in the present invention carries a mutation in at least one protein of the outer membrane. In certain preferred embodiments said host cell carries a mutation in the genes minA and/or minB. In most preferred embodiments, said prokaryotic host cell is Escherichia coli, most preferably Escherichia coli strain WCM104 or Escherichia coli strain WCM105. In other most preferred embodiments said prokaryotic host cell is produced as described in claim 1 of EP 0338410:
Process for the preparation of an E. coli strain for protein secretion of at least 140 mg of protein/I within 48 hours into the culture medium, characterized in that
(a) the structural gene of an exoprotein which is to be expressed is integrated, in a manner known per se, into a plasmid which is suitable for expression, to give a hybrid plasmid,
(b) an E. coli strain with a minA and/or minB mutation or an E. coli strain with a mutation in one protein or in several proteins of the outer membrane is transformed, in a manner known per se, with the hybrid plasmid which has been formed,
(c) the transformed E. coli strain is subjected, in a manner known per se, to mutagenesis, and
(d) where appropriate is subsequently exposed to substances acting on the cell wall,
(e) the cell material subjected to stages (c) and, where appropriate, (d) is subjected, in a manner known per se, to a screening for E. coli mutants with increased protein secretion compared with the E. coli strain used and
(f) where appropriate the E. coli mutant(s) with increased protein secretion obtained as in stage (e) is (are) rendered plasmid-free, in a manner known per se.

In particular embodiments, step (c) of claim 1 of EP 0338410 is carried out via chemical mutagenesis, for example with N-methyl-N′-nitro-N-nitrosoguanidine (MNNG). In other particular embodiments, D-cycloserine is used as substance acting on the cell wall in stage (d) in claim 1 of EP 0338410. In other particular embodiments, E. coli DS 410 (DSM 4513) or E. coli BW 7261 (DSM 5231) is used in accordance with EP 0338410 to generate a prokaryotic cell to be used in the present invention.

### DETAILED DESCRIPTION OF THE INVENTION

A “signal sequence”, “signal peptide” or “secretion signal sequence” as used herein refers to a stretch of amino acids within a polypeptide or protein which directs said polypeptide or protein, typically a newly synthesized polypeptide or protein, through a cellular membrane of a host cell. In prokaryotic cells, signal sequences typically direct polypeptides or proteins through the cytoplasmic membrane into the periplasmic space. Usually, the signal sequence is present at the N-terminus of a protein or polypeptide and facilitates its transport to the periplasm or into the culture medium of the host cell. Polypeptides and proteins comprising a signal sequence are referred to as “preprotein”. The signal sequence is generally removed from the N-terminus of the preprotein by enzymatic cleavage during translocation through the membrane, thereby producing the mature protein.

In Escherichia coli, signal sequences typically comprise between about 15 to 52 amino acids. Most signal sequences contain a positively charged N-terminal region (n-region), an apolar hydrophobic core (h-region) and a more polar C-terminal region (c-region). The c-region typically contains the cleavage site for signal peptidase. The determination of signal sequences is well known to the person skilled in the art. For example, signal sequences can be obtained from databases such as Swiss-Prot or GenBank or using annotated genome-wide data sets.

Signal sequence of the present invention may be homologous or heterologous origin. A homologous signal sequence is derived from the same species as the polypeptide or protein to which it is fused. In contrast, a heterologous signal sequence is derived from a different species. Any homologous or heterologous signal sequence may be cloned in association with a polypeptide or protein which is to be transported through a cellular membrane or into the periplasmic space by the host cell.

A suitable prokaryotic signal sequence may be obtained from genes encoding, for example, PhoE, MBP, LamB or OmpF OmpA, MalE, PhoA, STII and other genes.

Preferred signal sequences of the present invention are the following signal sequences of Escherichia coli, as well as functional derivatives thereof (all amino acids in one-letter code):

| Signal | |
|---|---|
| sequence of | Amino acid Sequence |
| MalE | MKIKTGARILALSALTTMMFSASALA |
| LamB | MMITLRKLPLAVAVAAGVMSAQAMA |
| PelB | MKYLLPTAAAGLLLLAAQPAMA |
| LivK | MKRNAKTIIAGMIALAISHTAMA |
| TorT | MRVLLFLLLSLFMLPAFS |
| TolB | MMNTRVWCKIIGMLALLVWLVSSPSVFAV |
| DsbA | MKKIWLALAGLVLAFSASA |
| Pac | MKNRNRMIVNCVTASLMYYWSLPALA |
| TorA | MNNNDLFQASRRRFLAQLGGLTVAGMLGPS |
| | LLTPRRATAAQA |
| PhoA | MKQSTIALALLPLLFTPVTKA |
| OmpA | MKKTAIAIAVALAGFATVAQA |

Certain preferred signal sequences are signal sequences of the SEC secretion pathways of Escherichia coli, such as the signal sequences of MalE, LamB, PelB, LivK, PhoA or OmpA. Other preferred signal sequences are signal sequences of the SRP secretion pathways of Escherichia coli, such as the signal sequences of TorT, TolB or DsbA. Yet other preferred signal sequences are signal sequences of the TAT secretion pathways of Escherichia coli, such as the signal sequences of Pac or TorA.

Other preferred signal sequences are signal sequences of pullulanases (Alpha-dextrin endo-1,6-alpha-glucosidase; EC 3.2.1.41), such as the signal sequences of the pullulanases of the following species:

Klebsiella aerogenes:
MLRYTCHALF LGSLVLLSG
Klebsiella pneumoniae:
MLRYTRNALV LGSLVLLSG
Thermoanaerobacter ethanolicus:
M FKRRTLGFL LSFLLIYTAV FGSMPVQFAK A
Thermoanaerobacter thermohydrosulfuricus:
MFKRRALGFL LAFLLVFTAV FGSMPMEFAK A
Thermoanaerobacter thermosulfurugenes:
MNKKLFTNRF ISFNMSLLLV LTAVFSSIPL HSVHA
Thermoanaerobacter saccharolyticum:
MYKKLFTKKF ISFVMSLLLV LTAAFSSMPF HNVYA
Thermotoga maritime:
MKTKLWLLLV LLLSALIFS

Also preferred are the following signal sequences, as well as functional derivatives thereof (all amino acids in one-letter code):

Heat-stable enterotoxin II of Escherichia coli (STII)

MKKNIAFLLA SMFVFSIATN AYA

phoE of Escherichia coli

MKKSTLALWMGIVASASVQA

Maltose binding protein (MBP) of Escherichia coli

MKIKTGARIL ALSALTTMMF SASALA

Alkaline phosphatase of Escherichia coli

MFSALRHRTA ALALGVCFIL PVHASSPKPG

Penicillinase (EC 3.52.6) of various species

e.g. Staphylococcus aureus: MKKLIFLIVIALVLSACNSNSSHA,
Escherichia coli: MKNTIHINFAIFLIIANIIYSSA,
Klebiella oxytoca: MLKSSWRKTALMAAAAVPLLLASG, or
Bacillus cereus: MKNKKMLKIGMCVGILGLSITSLVTFTGGALQVEAKEKTGQVK
Murein lipoprotein Lpp of Escherichia coli:

MKATKLVLGA VILGSTLLAG

Cyclomaltodextrin glucanotransferase of Klebsiella oxytoca: MKRNRFFNTSAAIAISIALNTFFCSMQTIA (SwissProt entry: P08704),
as well as functional derivatives thereof.

Also preferred are prokaryotic signal sequences selected from signal peptides of periplasmic binding proteins for sugars, amino acids, vitamins and ions, including signal peptides such as PelB (Erwinia chrysantemi, Pectate lyase precursor), PelB (Erwinia carotovora, Pectate lyase precursor), PelB (Xanthomonas campestris, Pectate lyase precursor), LamB (E. coli, Maltoporin precursor), MalE (E. coli, Maltose-binding protein precursor), Bla (E. coli, Beta-lactamase), OppA (E. coli, Periplasmic oligopeptide-binding protein), TreA (E. coli, periplasmic trehalase precursor), MppA (E. coli, Periplasmic murein peptide-binding protein precursor), BglX (E. coli, Periplasmic beta-glucosidase precursor), ArgT (E. coli, Lysine-arginine-ornithine binding periplasmic protein precursor), MalS (E. coli, Alpha-amylase precursor), HisJ (E. coli, Histidine-binding periplasmic protein precursor), XylF E. coli, D-Xylose-binding periplasmic protein precursor), FecB (E. coli, dicitrate-binding periplasmic protein precursor), OmpA (E. coli, outer membrane protein A precursor) PhoA (E. coli, Alkaline phosphatase precursor), OmpT (E. coli, outer membrane protein 2b), OmpC (E. coli, outer membrane protein 1b and the 17K antigen signal sequence of Rickettsia rickettsii.

The signal sequence may also be selected from any of the following signal sequences of E. coli, or any functional derivative thereof:

MNKNRGFTPLAVVLMLSGSLALTG
MTKHARFFLLPSFILISAALIAG
MKMRAVAVFTGMLTGVLSVAGLLSAGAYA
MNGSIRKMMRVTCGMLLMVMSGVSQA
MKRHLNTCYRLVWNHMTGAFVVASELARARGKRGGVAVALSLAAVTS
LPVLA
MKTLKNMRRKNLCITLGLVSLLSRGANA
MLKKSILPMSCGVLVMVMSGLLDA
MKTSSFIIVILLCFRIENVIA
MKIRRIVSTIAIALSVFTFAHA
MNKTLIAAAVAGIVLLASNAQA
MNKAYSIIWSHSRQAWIVASELARGHGFVLAKNTLLVLAVVSTIGNA
FA
MMYRIRNWLVATLLLLCTPVGA
MGSPSLYSARKTTLALAVALSFAWQAPVFA
MFKTTLCALLITASCSTFA
MKLAACFLTLLPGFAVA
MRKITQAISAVCLLFALNSSAVALA
MHKFTKALAAIGLAAVMSQSAMA
MKKVILSLALGTFGLGMAE
MKKSILALSLLVGLSTMSSYA
MKKVLIAALIAGFSLSATA
MKKLVLAALLASFTFGASA
MEFFKKTALAALVMGFSGAALA
MRRFLTTLMILLVVLVAGLSAL
MKWLCSVGIAVSLALQPALA
MRYIRLCIISLLATLPLAVHA
MSIQHFRVALIPFFAAFCLPVFA
MRLLPLVAAATAAFLVVA
MKNTIHINFAIFLIIANIIYSSA
MKTFAAYVITACLSSTALAS
MNLKKIAIASSVFAGITMALTCHA
MIKKASLLTACSTAFSAWA
MRKSLLAILAVSSLVFSSASFA
MEALGMIETRGLVALIEASDAMVKA
MRFLLGVLMLMISGSALA
MHKLFYLLSLLMAPFVANA
MKHKKKNRLVVAISVALIPYIG
MFRLNPFVRVGLCLSAISCAWPVLA
MSKRNAVTTFFTNRVTKALGMTLALMMTCQSAMA
MIKFSATLLATLIAASVNA
MKSKLIILLMLVPFSSFSTE
MKSKLIILLTLVPFSSFSTG
MKLLKVAAIAAIVFSGSALA
MKNKLLFMMLTILGAPGIAAA
MNTLLLLAALSSQITFN
MKRYLRWIVAAEFLFAAGNLHA
MRVKHAVVLLMLISPLSWA
MQRLFLLVAVMLLSG
MRKLFLSLLMIPFVAKA
MVKKAIVTAMAVISLFTLMG
MRLRKYNKSLGWLSLFAGTVLLSG
MFKSTLAAMAAVFALSALSPAAMA
MAVNLLKKNSLALVASLLLAGHVQA
MNTIFSARIMKRLALTTALCTAFISAAHA
MTQYSSLLRGLAAGSAFLFLFAPTAFA
MLLKRRLIIAASLFVFNLSSGFA
MKKQTQLLSALALSVGLTLSASFQAVA
MFVKLLRSVAIGLIVGAILLVAMPSLRS
MNKKVLTLSAVMASMLFGAAAHA
MASSALTLPFSRIAHA
MRISLKKSGMLKLGLSLVAMTVAASVQA
MKKIWLALAGLVLAFSASA
MKKGFMLFTLLAAFSGFAQA
MKRKVLLIPLIIFLAIA
MLKKILLLALLPAIAFA
MIKHVLLFFVFISFSVSA
MSSKKIIGAFVLMTGILSG
MAKVISFFISLFLISFPLYA
MSFKKIIKAFVIMAALVSVQAHA
MSHYKTGHKQPRFRYSVLARCVAWA
MKTILPAVLFAAFATTSAWA
MFFNTKHTTALCFVTCMAFSSSSIA
MKNITFIFFILLASPLYA
MKNITFIFFILLASPLYA
MNKVKFYVLFTALLSSLCAHG
MNKVKCYVLFTALLSSLYAHG
MRRVNILCSFALLFASHTSLA
MKFLPYIFLLCCGLWSTISFA
MKKTIGLILILASFGSHA
MKKTIGLILILASFGSHA
MLKKIIPAIVLIAGTSGVVNA
MLKKIISAIALIAGTSGVVNA
MVMSQKTLFTKSALAVAVALISTQAWS
MKKYVTTKSVQPVAFRLTTLSLVMSAVLGSASVIA
MSKRNAVTTFFTNRVTKALGMTLALMMTCQSAMA
MKKTMMAAALVLSALSIQSALA
MKITHHYKSLLSAIISVALFYSAA
MKKVTLFLFVVSLLPSTVLA
MLNIIHRLKSGMFPALFFLTSASVLA
MKKTLLAIILGGMAFATTNASA
MNKFISIIALCVFSSYANA
MKNKYNLLFFLFLLCYGDVALA
MKKLYKAITVICILMSNLQSA
MIKKVPVLLFFMASISITHA
MNKYPPLLTMLIIGIGSNAVA
MTPLRVFRKTTPLVNTIRLSLLPLAGLSFSAFA
MLAFIRFLFAGLLLVISHAFA
MNKKIHSLALLVNLGIYGVAQA
MRLAPLYRNALLLTGLLLSGIAAVQA
MARSKTAQPKHSLRKIAVVVATAVSGMSVYAQA
MSKRIALFPALLLALLVIVA
MSGLPLISRRRLLTAMALSPLLWQMNTAHA
MLSTQFNRDNQYQAITKPSLLAGCIALALLPSAAFA
MSNKNVNVRKSQEITFCLLAGILMFMAMMVAGRAEA
MSYLNLRLYQRNTQCLHIRKHRLAGFFVRLVVACAFA
MRNKPFYLLCAFLWLAVSHA
MKWCKRGYVLAAILALASATIQA
MKRVITLFAVLLMGWSVNAWSFA
MKSLFKVTLLATTMAVALHAPITFA
MLIIKRSVAIIAILFSPLSTA
MQKNAAHTYAISSLLVLSLTG
MIKFLSALILLLVTTAAQA
MRTLLAILLFPLLVQA
MKLAHLGRQALMGVMAVALVAGMSVKSFA
MKIKTLAIVVLSALSLSSTAALA
MKLKFISMAVFSALTLGVATNAS
MRMKKSALTLAVLSSLFSGYSLA
MKKLAIIGATSVMMMTGTAQA
MKKLAIMAAASMVFAVSSAHA
MKFKKTIGAMALTTMFVAVSASA
MKKLAIMAAASMVFAVSSAHA
MIKSVIAGAVAMAVVSFGAYA
MQKIQFILGILAAASSSSTLA
MKKTLIALAVAASAAVSGSVMA
MKKLAIMAAASMIFTVGSAQA
MKRLVFISFVALSMTAGSAMA
MIKSVIAGAVAMAVVSFGANA
MKKAFLLACVFFLTGGGVSHA
MKKTLIALAIAASAASGMAHA
MKKTLIALAIAASAASGMAHA
MKKTLIALAIAASAASGMAHA
MKLKKTIGAMALATLFATMGASA
MLKIKYLLIGLSLSAMSSYSLA
MKKNLLITSVLAMATVSGSVLA
MRIWAVLASFLVFFYIPQSYA
MFFGDGGQLLSDKSLTGSAGGGNNRMKFNILPLAFFIG
MIKPTFLRRVAIAALLSGSCFSAAA
MKSVLKVSLAALTLAFAVSSHA
MKLTLKNLSMAIMMSTIVMGSSAMA
MFFKKNLTTAAICAALSVAAFSAMA
MDCVMKGLNKITCCLLAALLMPCAGHA
MKKVLGVILGGLLLLPVVSNA
MGYKMNISSLRKAFIFMGAVAALSLVNAQSALA
MKKLVLSLSLVLAFSSATAAFA
MKKWLLAAGLGLALATSAQA
MKQWIAALLLMLIPGVQA
MKKSILFIFLSVLSFSPFA
MKKSILFIFLSVLSFSPFP
MKKNIAFLLASMFVFSIATNAYA
MKKLMLAIFISVLSFPSFS
MKKTTLALSRLALSLGLALSPLSATA
MTIEYTKNYHHLTRIATFCALLYCNTAFS
MFSALRHRTAALALGVCFILPVHA
MMISKKYTLWALNPLLLTMMAPAVA
MKLFKSILLIAACHAAQASA
MKLFKSILLIAACHAAQASA
MMITLRKLPLAVAVAAGVMSAQAMA
MNTKGKALLAGLIALAFSNMALA
MKRNAKTIIAGMIALAISHTAMA
MMKKIAITCALLSSLVASSVWA
MKKAKAIFLFILIVSGFLLVA
MKKITGIILLLLAVIILSA
MRKRFFVGIFAINLLVG
MCGKILLILFFIMTLSA
MKKITWIILLLLAAIILAA
MKKITGIILLLLAVIILAA
MKKITGIILLLLAAIILAA
MKKITGIILLLLAVIILAA
MKKITGIILLLLAAIILAA
MKIKTGARILALSALTTMMFSASALA
MKMNKSLIVLCLSAGLLASAPG
MNNEETFYQAMRRQGVTRRSFLKYCSLAATSLGLGAGMAPKIAWA
MNRRNFIKAASCGALLTGALPSVSHA
MMKMRWLSAAVMLTLYTSSSWA
MNKTAIALLALLASSASLA
MKGRWVKYLLMGTVVAMLAA
MFKRRYVTLLPLFVLLAA
MKKYLALALIAPLLIS
MKAKAILLASVLLVG
MARKWLNLFAGAALSFAVAGNALA
MKATKLVLGAVILGSTLLAG
MKLSRRSFMKANAVAAAAAAAGLSVPGVARA
MEFGSEIMKSHDLKKALCQWTAMLALVVSGAVWA
MKMRAVAVFTGMLTGVLSVTGLLSAGAYA
MDWLLDVFATWLYGLKVIAITLAVIMF
MLSTLRRTLFALLACASFIVHA
MKLTTHHLRTGAALLLAGILLAG
MAYSVQKSRLAKVAGVSLVLLLAA
MRFCLILITALLLAG
MSAGSPKFTVRRIAALSLVSLWLAG
MKKLTVAISAVAASVLMAMSAQA
MTRIKINARRIFSLLIPFFFFTSVHA
MSVLRSLLTAGVLASGLLWSLNGITATPAAQA
MNKGLLTLLLLFTCFAHAQVVDTWQFA
MKKTAIAIAVALAGFATVAQA
MKVKVLSLLVPALLVAGAANA
MMKRNILAVIVPALLVAGTANA
MKKLLPCTALVMCAGMA
MQTKLLAIMLAAPVVFSSQEASA
MRAKLLGIVLTTPIAISSFA
MKKIACLSALAAVLAFTAGTSVA
MTNITKRSLVAAGVLAALMAGNVALA
MFVTSKKMTAAVLAITLAMSLSA
MNKNMAGILSAAAVLTMLAG
MTMTRLKISKTLLAVMLTSAVATGSAYA
MKKRIPTLLATMIATALYSQQGLA
MRTLQGWLLPVFMLPMAVYA
MKNRNRMIVNCVTASLMYYWSLPALA
MQLNKVLKGLMIALPVMAIAA
MIKSVIAGAVAMAVVSFGVNNA
MKDRIPFAVNNITCVILLSLFCNA
MIRKKILMAAIPLFVISGADA
MKKIRGLCLPVMLGAVLMSQHVHA
MIRLSLFISLLLTSVAVL
MKKWFPAFLFLSLSGGNDALA
MRLRFSVPLFFFGCVFVHGVFA
MVVNKTTAVLYLIALSLSGFIHTFLRA
MIKSTGALLLFAALSAGQAIA
MRFSRFIIGLTSCIAFSVQA
MLIMPKFRVSLFSLALMLAVPFAPQAVA
MPRLLTKRGCWITLAAAPFLLFLAAWG
MNAKIIASLAFTSMFSLSTLLSPAHA
MKKSTLALVVMGIVASASVQA
MKKWSRHLLAAGALALGMSAAHA
MTALNKKWLSGLVAGALMAVSVGTLA
MKAILIPFLSLLIPLTPQSAFA
MKQSTIALALLPLLFTPVTKA
MNMFFRLTALAGLLAIAGQTFA
MRHSVLFATAFATLISTQTFA
MKKIRGLCLPVMLGAVLMSQHVHA
MIRLSLFISLLLTSVAVL
MKKWLPAFLFLSLSGCNDALA
MRLRFSVPLFFFGCVFVHGVFA
MVVNKTTAVLYLIALSLSGFIHTFLRA
MIKSTGALLLFAALSAGQAMA
MKRDGAMKITLLVTLLFGLVFLTTVG
MFKKGLLALALVFSLPVFA
MKVMRTTVATVVAATLSMSAFSVFA
MPRSTWFKALLLLVALWAPLSQA
MNMKKLATLVSAVALSATVSANAMA
MSGKPAARQGDMTQYGGSIVQGSAGV
MSGKPAARQGDMTQYGGSIVQGSAGV
MSGKPAARQGDMTQYGGSIVQGSAGV
MSGKPAARQGDMTQYGGPIVQGSAGV
MRKQWLGICIAAGMLAA
MRYLATLLLSLAVLITAG
MKAFWRNAALLAVSLLPFSSANA
MKQLWFAMSLVTGSLLFSANASA
MRQVLSSLLVIAGLVSGQAIA
MKLKFISMAVFSALTLGVATNASA
MVKDIIKTVTFSCMLAGSMFVTCHVCA
MAYSQPSFALLCRNNQTGQEFNS
MKLKAIILATGLINCIAFSAQA
MESINEIEGIYMKLRFISSALA
MMTKIKLLMLIIFYLIISASAHA
MRRVLFSCFCGLLWSSSGWA
MNMTKGALILSLSFLLAA
MEKAKQVTWRLLAAGVCLLTVSSVARA
MIKRVLVVSMVGLSLVG
MARTKLKFRLHRAVIVLFCLALLVALMQGA
MKRFSLAILALVVATGAQA
MVKSQPILRYILRGIPAIAVAVLLSA
MRKLTALFVASTLALGAANLAHA
MNKWGVGLTFLLAATSVMA
MSLSRRQFIQASGIALCAGAVPLKASA
MKNWKTLLLGIAMIANTSFA
MAISSRNTLLAALAFIAFQAQA
MLKKCLPLLLLCTAPVFA
MMNFNNVFRWHLPFLFLVLLTFRAAA
MKQALRVAFGFLILWASVLHA
MQMKKLLPILIGLSLSGFSSLSQA
MNNNDLFQASRRRFLAQLGGLTVAGMLGPSLLTPRRATA
MRVLLFLLLSLFMLPAFS
MNKALLPLLLCCFIFPASG
MKRILPLILALVAGMAQA
MKRRLWLLMLFLFAGHVPAASA
MKQTSFFIPLLGTLLLYG
MRCRGLIALLIWGQSVAA
MSLTKSLLFTLLLSAAAVQAST
MKLSMKSLAALLMMLNGAVMA
MKSPAPSRPQKMALIPACIFLCFAALSVQA
MSRFQRLTKYVAIGGGAALLLAGAAYLAGA
MSRILKRIAAGVVIAGVAALLLAAGGYAAG
MMPRIKPLLVLCAALLTVTPAASA
MKMKKLMMVALVSSTLALSG
MMKTKKLMMVALVSSTLALSG
MKHNVKLMAMTAVLSSVLVLSG
MKTKKLMMVALVSSTLALSG
MKKTLLAAGAVLALSSSFTVNA
MKPLHYTASALALGLALMGNAQA
MAMAVICLTAASGLTSAYA
MAMKKLLIASLLFSSATVYG
MGRISSGGMMFKAITTVAALVIATSAMA
MKLLQRGVALALLTTFTLASETALA
MKLRLSALALGTTLLVG
MKKMLLATALALLITG
MMKSKMKLMPLLVSVTLISG
MKIKNILLTLCTSLLLTNVAAHA
MKSVFTISASLAISLMLCCTAQA
MKTFFRTVLFGSLMAVCANS
MQRRDFLKYSVALGVASALPLWSRAVFA
MKTIFRYILFLALYSCCNTVS
MYKQAVILLLMLFTASVSA
MMTFKNLRYGLSSSVVLAASLFSVLSYA
MIKTTPHKIVILMGILLSPSVFA
MSKKLGFALSGLMLAMVAGTASA
MAKSLFRALVALSFLAPLWLNA
MAFKFKTFAAVGALIGSLALVG
MPERVLNFRALFLHGLYKMDKPKAYCRLFLPSFLLLSA
MSLNFSAFSDVLSPLAECAPTFA
MRKIALILAMLLIPCVSFA
MSLPSIPSFVLSGLLLI
MNSKKLCCICVLFSLLAG
MPLRRFSPGLKAQFAFGMVFLFVQPDASA
MKKHLLPLALLFSGISPA
MKKKVLAIALVTVFTGMGVAQA
MKIISKMLVGALALAVTNVYA
MHSWKKKLVVSQLALACTLAITSQANA
MFKKILFPLVALFMLAG
MKLVHMASGLAVAIALAA
MKYSSIFSMLSFFILFA
MRKFIFVLLTLLLVSPFSFA
MNKEQSADDPSVDLIRVKNMLNSTISMS
MDLNEASLNAASTRA
MQLRKPATAILALALSAGLAQA
MELYREYPAWLIFLRRTYAVA
MKRVLFFLLMIFVSFGVIA
MKKLILIAIMASGLVA
MKTNRSLVVIVSLITATLLLTA
MFKGQKTLAALAVSLLFTAPVYA
MSSNFRHQLLSLSLLVGIAAPWAAFA
MPFTSSGGEMSAGKGLLLVICLLFLPLKSAMA
MDTVNIYRLSFVSCLVMAMPCAMA
MNTFSVSRLALALAFGVTLTA
MGKAVIAIHGGAGAISRA
MNMKLKTLFAAAFAVVGFCSTA
MIMKNCLLLGALLMGFTGVAMA
MRYSKLTMLIPCALLLSA
MPARHLYFIMTNTWNRLALLIFAVLSLLVAGELQA
MITMKKSVLTAFITVVCATSSVMA
MKTCITKGIVTVSLTAILLSCSSAWA
MYRTHRQHSLLSSGGVPSFIGGLVVFVSAA
MKKNIFKFSVLTLAVLSLTA
MQYKDENGVNEPSRRRLLKVIGALALAGSCPVAHA
MLRNGNKYLLMLVSIIMLTA
MYSSSRKRCPKTKWALKLLTAAFLAA
MYPVDLHMHTVASTHA
MKKSLLGLTFASLMFSAGSAVA
MMIKTRFSRWLTFFTFAAAVALA
MNMMRIFYIGLSGVGMMFSSMA
MKIKSIRKAVLLLALLTSTSFA
MSTTHNVPQGDLVLRTLAMPADTNA
MKKLTVAALAVTTLLSGSAFA
MLELLFLLLPVAAAYG
MKILYSFLLLPFFSCA
MKKENYSFKQACAVVGGQSAMA
MMMKKTLLLCAFLVGLVSSNVMA
MKKLALILFMGTLVSFYADA
MAMAAVCGTSGIASLFSQAAFA
MTVSSHRLELLSPARDAAIA
MKFQAIVLASFLVMPYALA
MNRIYRVIWNCTLQVFQA
MARINRISITLCALLFTTLPLTPMAHA
MTKLKLLALGVLIATSAGVAHA
MEKNMKKRGAFLGLLLVSA
MEIRIMLFILMMMVMPVSYA
MLRMTPLASAIVALLLGIEAYA
MAAIPWRPFNLRGIKMKGLLSLLIFSMVLPA
MKRSIIAAAVFSSFFMSAGVFA
MNKYWLSGIIFLAYGLASPAFS
MEFYMKAFNKLFSLVVASVLVFSLAG
MYDFNLVLLLLQQMCVFLVIAWLMS
MPLLKLWAGSLVMLAAVSLPLQA
MQMIVRILLLFIALFTFGVQA
MRIIFLRKEYLSLLPSMIASLFS
MSDLLCSAKLGAMTLALLLSATSLSALA
MPDHSLFRLRILPWCIALAMSGSYSSVWA
MRKYIPLVLFIFSWPVLCA
MIKHLVAPLVFTSLILTG
MKIRSLSRFVLASTMFASFTASA
MSAFKKSLLVAGVAMILSNNVFA
MPKLRLIGLTLLALSATAVSHA
MKKFAAVIAVMALCSAPVMA
MKLITAPCRALLALPFCYAFS
MKIKTILTPVTCALLISFSAHA
MKFKTNKLSLNLVLASSLLAASIPAFA
MELLCPAGNLPALKA
MLKKTLLAYTIGFAFSPPANA
MNNVKLLIAGSAFFAMSAQA
MRRLPGILLLTGAALVVIA
MQCGISDGLPGFSYADADGKFS
MKLNIFTKSMIGMGLVCSALPALA
MKRLLILTALLPFVGFA
MDKSKRHLAWWVVGLLAVA
MNRRRKLLIPLLFCGAMLTA
MQGTKIRLLAGGLLMMATAGYVQA
MNVLRSGIVTMLLLAAFSVGA
MKRKLFWICAVAMGMSAFPSFMTQA
MKKRVYLIAAVVSGALAVSG
MKLRSVTYALFIAGLAAFSTSSLA
MLINRNIVALFALPFMASATA
MDLLIILTYVAFAWA
MKACLLLFFYFSFICQLHG
MLLHILYLVGITAEA
MMNVSEKMEHFDVAIIGLGPAGSALA
MDFSIMVYAVIALVGVAIGWLFA
MMRIVTAAVMASTLAVSSLSHA
MKPGCTLFFLLCSALTVTTEAHA
MKNRLLILSLLVSVPAFA
MKASLALLSLLTAFTSHS
MKKVLYGIFAISALAATSAWA
MVILPLWRRVVKRPALILICLLLQA
MIKQTIVALLLSVGASSVFA
MKKRHLLSLLALGISTA
MKKIIALMLFLTFFAHA
MNKYLKYFSGTLVGLMLSTSAFA
MMNRVIPLPDEQATLDLGERVAKA
MASSSLIMGNNMHVKYLAGIVGAALLMAGCSSS
MRLIITFLMAWCLSWGAYA
MGILKSLFTLGKSFISQA
MFSRVLALLAVLLLSANTWA
MKFMKKAKILSGVLLLCFSSPLISQA
MNAIISPDYYYVLTVAGQSNA
MMMKTVKHLLCCAIAASALISTGVHA
MTILSLSRFMLAGVLLASFNASA
MKKALQVAMFSLFTVIGFNAQA
MKKIALIIGSMIAGGIISAAGFTWVAKA
MHRQSFFLVPLICLSSALWA
MKKNSYLLSCLAIAVSSA
MNKLQSYFIASVLYVMTPHAFA
MRPLILSIFALFLAG
MKRASLLTLTLIGAFSAIQAAWA
MEISFTRVALLAAALFFVG
MPTKMRTTRNLLLMATLLGSALFARA
MKIILLFLAALASFTVHA
MKTIFTVGAVVLATCLLSG
MKLMRYLNTKNIIAAGVLLSCMSSIAWG
MRYLLIVITFFMGFSSLPAWA
MEGSRMKYRIALAVSLFALSAGS
MNKVTKTAIAGLLALFAGNAAA
MSKRTFAVILTLLCSFCIGQALAGG
MPQRHHQGHKRTPKQLALIIKRCLPMVLTGSGMLCTTANA
MKRAPLITGLLLISTSCAYA
MKALSPIAVLISALLLQGCVAAA
MFKRLMMVALLVIAPLSAATA
MQTKKNEIWVGIFLLAALLAALFVCLKA
MKKINAIILLSSLTSASVFA
MTLRKILALTCLLLPMMASA
MRYIRQLCCVSLLCLSGSAVA
MWKRLLIVSAVSAAMSSMALA
MRVIMKPLRRTLVFFIFSVFLCGTVS

Also within the scope of the present invention are all functional derivatives of the signal sequences described herein. “Functional derivates” as used in this context refers to any signal sequence which is based on any of the naturally occurring signal sequences described herein, but which was intentionally or unintentionally modified, thereby still fulfilling its function of directing polypeptides or proteins into the prokaryotic periplasmic space or through a cellular membrane. Intentional modifications include purposely introduced amino acid substitution, such as by site-directed mutagenesis of the respective nucleic acid encoding for said amino acids, and purposely introduced insertions or deletions. Unintentional modifications, such as point mutations, insertions or deletions, may occur during passage of the signal sequence on the vector or the genome of the host cell.

Suitable eukaryotic signal sequences may be obtained from genes encoding, for example, gp70 from MMLV, Carboxypeptidase Y, KRE5 protein, Ceruloplasmin precursor, Chromoganin precursor, beta-hexosaminidase a-chain precursor and other genes.

The signal sequences to be employed in the present invention can be obtained commercially or synthesized chemically. For example, signal sequences can be synthesized according to the solid phase phosphoramidite triester method described, e.g., in Beaucage & Caruthers, Tetrahedron Letts. 22:1859-1862 (1981), using an automated synthesizer, as described in Van Devanter et. al., Nucleic Acids Res. 12:6159-6168 (1984).

A “promoter” as used herein refers to a nucleic acid molecule encoding a regulatory sequence controlling the expression of a nucleic acid molecule of interest. Promoters which may be used include, but are not limited to the SV40 early promoter region, the promoter contained in the 3′ long terminal repeat of the RSV virus, the herpes thymidine kinase promoter, the tetracycline (tet) promoter, β-lactamase promoter or the tac promoter. As used herein, “operably linked” means the association of two or more DNA fragments in a DNA construct so that the function of one, e.g. protein-encoding DNA, is affected by the other, e.g. a promoter. For example, a promoter is operably linked to a gene of interest if the promoter regulates or mediates transcription of the gene of interest in a cell.

An “immunoglobulin” or “Ig” as used herein refers to a typical protein belonging to the class IgG, IgM, IgE, IgA, or IgD (or any subclass thereof), and includes all conventionally known antibodies and functional fragments thereof. Immunoglobulins typically comprise four polypeptide chains, two identical heavy chains and two identical light chains. The heavy chains typically comprise a variable region (V_{H}) and a constant region (C_{H}), which comprises a C_{H1}, a C_{H2} and a C_{H3} region. The light chains typically comprise a variable region (V_{L}) and one constant region (C_{L}). Immunoglobulins of the present invention comprise at least a portion of a C_{H3} region. An immunoglobulin comprising the entire heavy chains and light chains is referred to as “full-length immunoglobulin”.

A “functional fragment” of an immunoglobulin hereby is defined as a fragment of an immunoglobulin that retains an antigen-binding region and which comprises at least a portion of a C_{H3} region. A “portion of a C_{H3} region” is hereby defined as comprising at least one amino acid belonging to said C_{H3} domain of the heavy chain constant region. Typically the heavy chain of a functional fragment comprises a variable region (V_{H}) and a constant region (C_{H}), which comprises a C_{H1}, a C_{H2} and at least a portion of a C_{H3} region. A functional fragment of an immunoglobulin may also comprise minor deletions or alterations in the V_{H} and/or V_{L} region, provided antigen-binding is maintained.

An “antigen-binding region” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs.

The terms “immunoglobulin” and “antibody” are used interchangeably in the broadest sense as a protein, which can bind to an antigen, comprising at least an antibody variable region, preferably a V_{H} region and optionally also a V_{L} region. Numerous known antibody sequences are listed, and the conserved structure of antibody variable regions is discussed in Kabat et al. (1991), Sequences of Immunological Interest, National Institutes of Health, Bethesda, Md. A variable region comprises three complementarity determining regions (CDRs) and four framework regions (FRs) arranged in the following order: FR1 CDR1 FR2 CDR2 FR3 CDR3 FR4. FRs are conserved in sequence relative to CDRs.

The terms “protein” and “polypeptide” are art recognized and used herein interchangeably.

A “host cell” as used herein refers to any prokaryotic cell used in the present invention to produce immunoglobulins, preferably full-length immunoglobulins, thereby secreting the immunoglobulins produced into the culture medium. Most preferred host cells are prokaryotic cells, even more preferred procaryotic cells carrying a mutation in at least one protein of the outer membrane. Particularly preferred as host cells are Gram-negative prokaryotes, most preferably Escherichia coli. In certain embodiments said Escherichia coli carries a mutation in the gene minA and/or minB. In other embodiments said Escherichia coli is Escherichia coli strain WCM104. In yet other embodiments said Escherichia coli is Escherichia coli strain WCM105.

### BRIEF DESCRIPTION OF FIGURES

FIG. 1:

The IgG construct with Cys (C) to Ser (S) mutations lacking inter chain disulfide bonds designed for expression in E. coli is shown in panel A. As a contrast the natural IgG construct with disulfide bonds between heavy and light chain as well as inter heavy chain disulfide bonds in the hinge region is depicted in panel B.

FIG. 2:

FIG. 2 shows the architecture of the IgG1 bicistronic expression cassettes in the expression vectors in detail. Light chain and heavy chain are in tandem orientation under control of one Ptac promoter. Translation initiation regions (SD-SEQ) are located in front of the coding regions of light and heavy chains. Signal sequences direct transport of light and heavy chain into bacterial periplasm.

FIG. 3:

Binding of E. coli WCM105 produced MOR01555 IgG to ICAM-1 coated onto ELISA plates. Serial dilutions of bacterial culture medium were applied to ELISA plates coated with ICAM-1. Detection of IgG was performed using a goat-anti human IgG, F(ab′)₂ fragment specific peroxidase conjugated antibody (Jackson Immuno Research), at a dilution of 1:10.000 in BPBS.

FIG. 4:

Result of Western-Blot analysis of bacterial culture medium, column flow-through and purified samples of E. coli WCM105 expressions of MOR01555 IgG and for comparison of a MOR01555_Fab_MH. MOR01555_IgG was purified via Protein A chromatography, the MOR01555 Fab_MH sample was applied to standard IMAC chromatography. Bands representing Ig heavy chain, Fab_MH heavy chain fragment and the Ig light chain are indicated on the right. A triangle (∇) points to an additional band detected in the purified IgG sample.

### EXAMPLES

The present invention can be better understood with reference to the following examples, which are not intended to limit the scope of the invention as described above.

### Example 1

### Cloning of IgG Expression Vectors

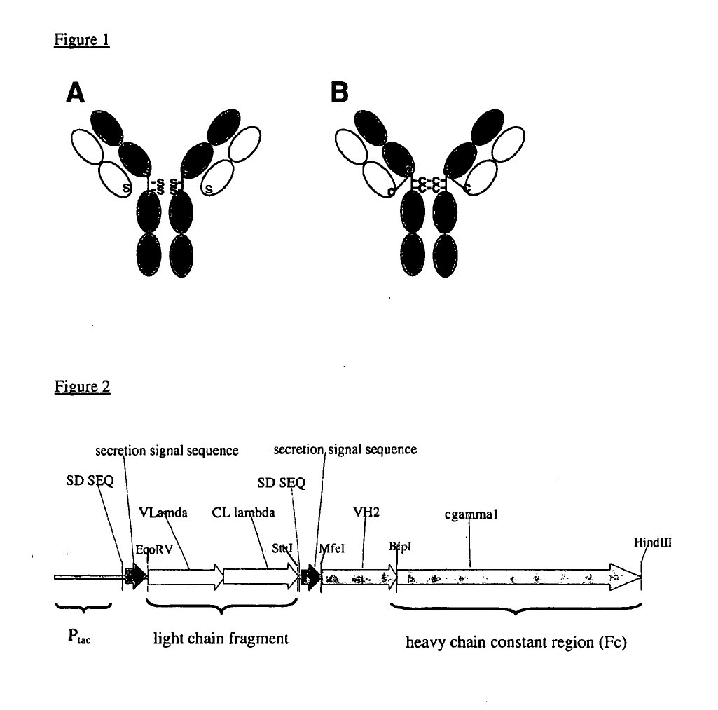

In order to express full length IgG in E. coli the cDNA sequence coding for the human IgG1 constant region (NCBI Nucleotide entry: Y14737 (GI: 2765424); including CH1, hinge, CH2 and CH3 domains) was de novo synthesized by Geneart AG (Regensburg, Germany) with a codon usage optimized for expression in E. coli (GeneOptimizer sequence optimization technology of Geneart (Regensburg, Germany)). Moreover, cysteine residues were mutated to serine residues to avoid disulfide bond formation between the heavy and the light chain and to avoid formation of inter-heavy chain disulfide bonds (FIG. 1).

The optimized heavy chain IgG1 constant region described above was cloned into the vector pEX-FabA-mut-Hind/Xba via the restriction sides BlpI and HindIII, yielding an IgG expression vector designated pEX_IgG MOR01555. This IgG expression vector contains an expression cassette for MOR01555 human IgG1 lambda with light chain and heavy chain in tandem orientation under control of the P_{tac} promoter. MOR01555 is an antibody specific for ICAM-1.

In order to simplify subcloning of other antibody candidates, vector backbone of pEX-FabA-mut-Hind/Xba expression vector was modified in several restriction sites. The resulting pEX_MV2_MOR01555_Fab_FS vector was used to generate pEX_MV2_MOR01555_IgG1 by subcloning of the optimized heavy chain IgG constant region described above via BlpI and HindIII.

In the same manner an expression vector construct containing an expression cassette for MOR03207 was generated, designated pEX_MV2_IgG MOR03207_IgG1. MOR03207 is an antibody specific for lysozyme. FIG. 2 shows the architecture of the IgG expression cassette generated. With exception of VH and VL sequences, the elements of the IgG expression cassette were identical in pEX_IgG MOR01555, pEX_MV2_MOR01555_IgG1 and pEX_MV2_MOR03207_IgG1. The sequences of all IgG expression cassettes were confirmed via sequencing using appropriate primers.

The signal sequence fused to the N-terminus of the variable domain of the heavy chain was the same for all constructs, i.e. the phoA signal sequence (MKQSTIALALLPLLFTPVTKA).

Various signal sequences were fused to the N-terminus of the variable domain of the light chain:

| Signal | |
|---|---|
| sequence of | Amino acid sequence |
| MalE (E. coli) | MKIKTGARILALSALTTMMFSASALA |
| Lamb (E. coli) | MMITLRKLPLAVAVAAGVMSAQAMA |
| PelB (E. coli) | MKYLLPTAAAGLLLLAAQPAMA |
| LivK (E. coli) | MKRNAKTIIAGMIALAISHTAMA |
| TorT (E. coli) | MRVLLFLLLSLFMLPAFS |
| TolB (E. coli) | MMNTRVWCKIIGMLALLVWLVSSPSVFAV |
| DsbA (E. coli) | MKKIWLALAGLVLAFSASA |
| Pac (E. coli) | MKNRNRMIVNCVTASLMYYWSLPALA |
| TorA (E. coli) | MNNNDLFQASRRRFLAQLGGLTVAGMLGP |
| | SLLTPRRATAAQA |
| PhoA (E. coli) | MKQSTIALALLPLLFTPVTKA |
| OmpA (E. coli) | MKKTAIAIAVALAGFATVAQA |

Signal sequences from all three secretion pathways are included in this selection: SEC (malE, lamB, pelB, livK, phoA, ompA), SRP (torT, tolB, dsbA), TAT (pac, torA).

DNA fragments containing all signal sequences were generated by de-novo DNA synthesis (Geneart, Regensburg, Germany) and cloned into vectors pEX_MV2_MOR03207_IgG1 or pEX_MV2_MOR01555_IgG1 using restriction enzymes EcoRI and EcoRV. The nucleic acid sequences encoding the signal sequences were selected as follows:

| Signal | |
|---|---|
| sequence of | Nucleic acid sequence |
| MalE | ATGAAAATAAAAACAGGTGCACGCATCCTCGC |
| (E. coli) | ATTATCCGCATTAACGACGATGATGTTTTCCG |
| | CCTCGGCTCTCGCC |
| Lamb | ATGATGATTACTCTGCGCAAACTTCCTCTGGC |
| (E. coli) | GGTTGCCGTCGCAGCGGGCGTAATGTCTGCTC |
| | AGGCAATGGCT |
| PelB | ATGAAATACCTATTGCCTACGGCAGCCGCTG |
| (E. coli) | GATTGTTATTACTCGCGGCCCAGCCGGCCAT |
| | GGCC |
| LivK | ATGAAACGGAATGCGAAAACTATCATCGCAG |
| (E. coli) | GGATGATTGCACTGGCAATTTCACACACCGC |
| | TATGGCT |
| TorT | ATGCGCGTACTGCTATTTTTACTTCTTTTCC |
| (E. coli) | CTTTTCATGTTGCCGGCATTTTCG |
| TolB | ATGAAGCAGGCATTACGAGTAGCATTTGGTT |
| (E. coli) | TTCTCATACTGTGGGCATCAGTTCTGCATGC |
| | T |
| DsbA | ATGAAAAAGATTTGGCTGGCGCTGGCTGGTT |
| (E. coli) | TAGTTTTAGCGTTTAGCGCATCGGCG |
| Pac | ATGAAAAATAGAAATCGTATGATCGTGAACT |
| (E. coli) | GTGTTACTGCTTCCCTGATGTATTATTGGAG |
| | CTTACCTGCACTGGCT |
| TorA | ATGAACAATAACGATCTCTTTCAGGCATCAC |
| (E. coli) | GTCGGCGTTTTCTGGCACAACTCGGCGGCTT |
| | AACCGTCGCCGGGATGCTGGGGCCGTCATTG |
| | TTAACGCCGCGACGTGCGACTGCGGCGCAAG |
| | CG |
| PhoA | ATGAAACAAAGCACTATTGCACTGGCACTCT |
| (E. coli) | TACCGTTGCTCTTCACCCCTGTTACCAAAGC |
| | C |
| OmpA | ATGAAAAAGACAGCTATCGCGATTGCAGTGG |
| (E. coli) | CACTGGCTGGTTTCGCTACCGTAGCGCAGGC |
| | C |

Constructs were transformed into E. coli WCM105 and expression in 20 ml scale was performed as described below. Two parallel expression cultures of each construct were inoculated from corresponding seed cultures.

### Example 2

### IgG Expression in Escherichia coli

IgG expression vectors (generated as described in Example 1) were transformed into E. coli WCM105 (E. coli WCM105 can be prepared from E. coli DS410, as described in EP0338410B1, which is hereby incorporate by reference in its entirety) by electroporation. Bacteria were plated onto 2xYT or V67 plates containing 10 or 20 μg/ml Tetracycline (Tet) and grown overnight at 37° C. Seed cultures were inoculated from transformed plates into 2xYT or V67 medium containing 20 μg/ml Tetracycline and grown overnight at 30° C. and 250 rpm. 20 ml expression cultures in 2xYT or V67 medium supplemented with 20 mg/ml Tetracycline, trace elements, 0.3% glucose and 1% lactose were inoculated with pre-culture to a start OD₆₀₀ of 0.1 and grown in 100 ml non-baffled shake flasks at 30° C. and 200-250 rpm for 72 h. Cultures were harvested at 5000×g for 10 min and supernatant sterile filtered. Two parallel expression cultures of each construct were inoculated from a corresponding seed culture.

### Example 3

### Proof of Concept Via Protein a Affinity Purification

In order to demonstrate that the IgG's were actually secreted into the culture medium of the host cells, the culture supernatant was subjected to purification via a Protein A affinity column. For this experiment E. coli WCM105 transformed with pEX_MV2_IgG MOR03207_IgG1 was used.

### Experimental Procedure:

After growth of the expression cultures as described in Example 2, 17 ml of the each respective culture media was adjusted with 1.9 ml 10×PBS for purification via a GX-274 Gilson robot
0.2 ml Protein A FF (GE Healthcare), packed in 1 ml Varian plastic columns were used
columns were equilibrated with 10 column volumes 1×PBS pH 7.2 and 18.5 ml adjusted supernatants were loaded onto the columns
Protein A columns were washed with 10 column volumes 1×PBS
Elution was done with 100 mM glycine pH 3 in one 450 μl fraction adjusted with 50 μl 1M Tris pH 8 after pre-elution with one column volume
fractions of all columns were measured at UV280 nm with a Nanodrop photometer against elution buffer
the column was regenerated with 10 column volumes of 0.5M NaCl pH 2

| | | A260/A280 | Expression rate |
|---|---|---|---|
| Experiment No. | signal peptid | ratio | mg/l |
| 1b1 | malE | 0.69 | 15.0 |
| 1b2 | malE | 0.66 | 12.1 |
| 2b1 | lamB | 0.73 | 18.5 |
| 2b2 | lamB | 0.65 | 20.3 |
| 3b1 | pelB | 0.61 | 18.8 |
| 3b2 | pelB | 0.84 | 22.4 |
| 4b1 | livK | 0.62 | 12.1 |
| 4b2 | livK | 0.85 | 18.8 |
| 6b1 | torT | 0.96* | 27.4* |
| 6b2 | torT | 0.62 | 15.6 |
| 7b1 | tolB | 1.10* | 32.4* |
| 7b2 | tolB | 1.10* | 29.4* |
| 8b1 | dsbA | 0.71 | 17.9 |
| 8b2 | dsbA | 0.61 | 15.9 |
| 9b1 | pac | 0.61 | 18.8 |
| 9b2 | pac | 0.60 | 17.9 |
| 10b1 | torA | 0.67 | 15.3 |
| 10b2 | torA | 0.93* | 19.4* |
| 11b1 | phoA | 1.33* | 14.4* |
| 11b2 | phoA | 0.70 | 2.4 |
| 12b1 | ompA | 0.68 | 19.1 |
| 12b2 | ompA | 0.61 | 19.1 |

As can be seen in Table 1, immunoglobulins could be eluted from the Protein A affinity column with all signal sequences tested. The A260/A280 ration gives an indication of the contamination of the protein fractions with nucleic acids. Ratios greater than 0.9 give rise to inaccuracies in the determination of the protein content of the respective fractions and the expression rates calculated therefrom. Such measurements are highlighted with an asterisk (*). Based on the protein contents of the individual fractions the expression rate was extrapolated for a culture volume of one litre (last column of Table 1). The expression rates determined are surprisingly high, and higher than the values typically received for respective immunoglobulins produced in eukaryotic systems.

The entire experiment was repeated and again two parallel expression cultures of each construct was inoculated from corresponding seed cultures. Results of this second set of experiments generally confirmed the expression rates determined in the first set of experiments.

All fractions eluted from the Protein A affinity column were also subjected to SDS-PAGE. The heavy and the light chain of the immunoglobulins are clearly visible. With the exception of some degradation products of the heavy chain in some fractions, no prominent other bands could be seen on the gel.

In alternative experiments E. coli WCM105 transformed with pEX_MV2_IgG MOR01555 was used. in the following set up:

10 ml plastic columns (Pierce) packed with 1 ml 50% rProtein A Sepharose™ Fast Flow (GE) were used for gravity flow purification. Supernatants (5 ml culture medium) were adjusted with 10× Running Buffer (RB) and loaded onto columns equilibrated with 5 column volumes (CV) RB. Column was washed with 10 CV RB and elution done with 5 CV elution buffer (EB). Fractions of 250 μl were collected and neutralized with 1/10 of neutralization buffer. For regeneration 5 CV were used and the column then re-equilibrated with 10 CV RB. Recipes for buffers are outlined below:

### Running Buffer: 148 mM PBS

Elution Buffer: 100 mM glycine pH 3

### Neutralization Buffer: 3 M Tris pH 8

### Regeneration Buffer: 0.5 M NaCl pH 2

Samples were analysed under denaturing, reducing conditions using 15% Tris-HCl Criterion Precast gel (BioRad, 26-well). Running conditions: 10 min 100 V, 50 min 200 V; Running buffer: 25 mM Tris, 192 mM Glycine, 0.1% SDS.

The results with the lysozame-binding immunoglobulin MOR03207 could be confirmed.

In summary, these experiments clearly showed and confirmed the surprising finding that functional IgG's can be produced in prokaryotic cells, and that said IgG's can be secreted into the culture medium via numerous signal sequences.

### Example 4

### ICAM-1 specific Elisa to Confirm the Presence of Functional IgG's in Bacterial Culture Medium

Some IgG's were characterized in more detail. In one experiment the presence of functional IgG's in bacterial culture medium was confirmed via ELISA. An antibody containing the expression cassette for MOR01555 was used in this experiment.

A black 96-well Maxisorp microtiter plate (Nunc) was coated over night at 4° C. with 50 μl/well of 0.5 μg/ml human ICAM-1-Fc fusion protein (R&D Systems). The ELISA plate was blocked with 100 μl/well PBS+2% BSA (BPBS) for 1-2 h at RT. Purified reference Fab-dHLX, test- and QC samples were appropriately pre-diluted and applied in 8 serial 2-fold dilutions. Per ELISA plate 2 series of reference probe (starting with 20 ng/ml for Fab-A dHLX) a High-QC and Low-QC sample and test samples were applied. QC samples were spiked into mock control produced in the appropriate medium. The concentration of QC samples was adjusted to the highest and lowest expected test sample concentration. Samples were diluted in BPBS using polypropylene microtiter plates (Nunc). After 5 washing cycles with PBS+0.05% Tween20 (PBST), 50 μl/well of diluted samples were transferred to the ELISA plate and incubated for 1-2 h at RT. The plate was washed again as described above and 50 μl/well of goat-anti human IgG, F(ab′)₂ fragment specific peroxidase conjugated antibody (Jackson Immuno Research), diluted 1:10.000 in BPBS was added. After washing, 50 μl/well QuantaBlu peroxidase-substrate solution (Pierce) was added and fluorescence was measured at 320/430 nm Ex/Em using a SpectraFluor Plus instrument (Tecan).

Data were evaluated with XL-fit software (IDBS, Emeryville, USA) using a 4-parameter logistic fit model. Sample concentrations were calculated form the average of all dilutions within the calibration range. Dilution linearity of reference sample and recovery of QC samples were checked in order to assess data quality of each test run.

This experiment also is a rough measure for the IgG titer in the bacterial supernatant.

Investigation of bacterial culture medium in ELISA revealed specific ICAM-1 binding activity which could be titrated by a serial dilution, indicating that a significant amount of functional IgG was secreted into the bacterial culture medium (FIG. 3).

By using a Fab_dHLX standard of known concentration the IgG titer in the bacterial supernatant could be roughly determined to be about 12.5 μg/ml.

In equivalent experiments the presence of functional IgG's is demonstrated for the other constructs generated in Example 1.

### Example 5

### Western Blots to Confirm the Production of Full Length IgG's

The presence of full length IgG heavy and light chains in bacterial culture medium was also confirmed via Western Blots. Polypeptides of the culture medium were separated via SDS-PAGE as described in Example 3. Proteins were then blotted on a nitrocellulose membrane for 1 h at 100 V using a BioRad Wet-Blot system. The membrane was blocked with 3% milk powder in TBS containing 0.1% Tween 20. For detection of heavy and light chains the following antibodies and conditions were applied:

Detection of heavy chain: Sheep anti-human IgG, Fd specific (The Binding Site) 1:10000 and anti-sheep IgG-AP conjugate (Sigma) 1:10.000 as a detection antibody.

Detection of light chain: Anti-human lambda light chain, AP conjugated (Sigma) 1:1000.

The blot was developed using Fast BCIP/NBT substrate (Sigma).

An antibody containing the expression cassette for MOR01555 was used in this experiment. For a comparison, respective samples of MOR01555 Fab_MH were investigated in parallel to the IgG samples.

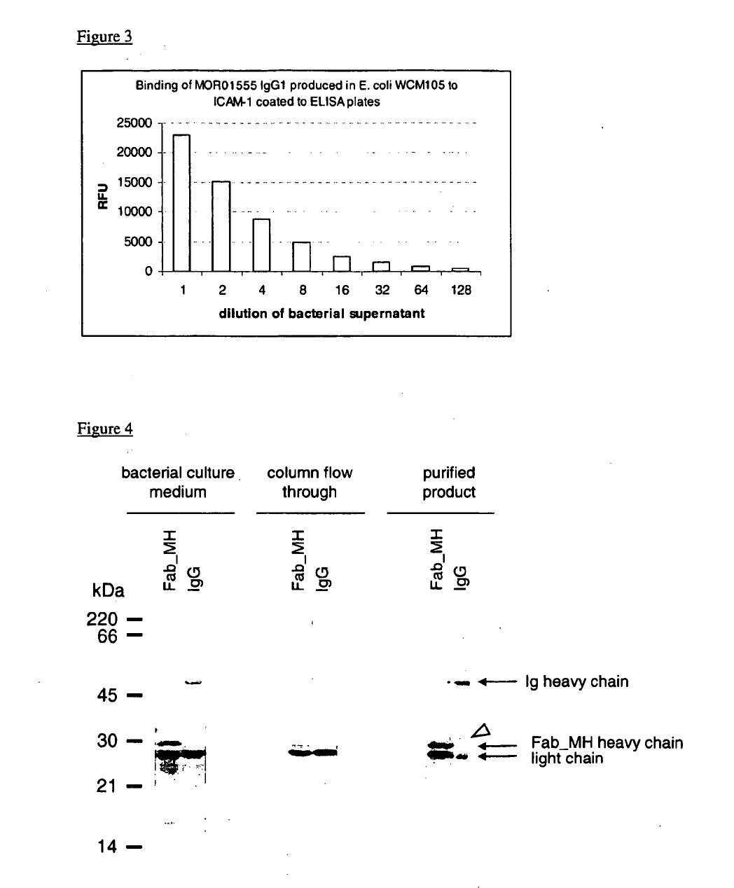

Light chain and heavy chain were sequentially detected using primary antibodies with respective specificities (FIG. 4).

The Western-Blot in FIG. 4 shows that both Ig light and full length heavy chain are present in the MOR01555 IgG sample in bacterial culture medium at the expected size. However, excess of light chain over heavy chain was approx. 10-20 fold in the raw sample.

Though a significant excess of light chain could also be found in the MOR01555 Fab_MH sample, heavy to light chain ratio was much more balanced than in the IgG sample. This finding indicates, that production or secretion of full length Ig heavy chain is a somewhat limiting factor and overall IgG yield in supernatant might be drastically increased by improving secretion of Ig full length heavy chain.

Nearly exclusively light chain was detected in the column flow through samples. This finding was expected due to the excess of light chain in the bacterial culture medium and since purification via Protein A (MOR01555 IgG) and IMAC (MOR01555 Fab_MH) depends on sequences in the heavy chain. In contrast, both purified samples revealed a very balanced amount (˜1:1 relationship) of heavy and light chain indicating that pairing of heavy and light chain functions normally during secretion process or in bacterial culture medium.

Altogether, these data clearly show that full length, functional IgG can be expressed in prokaryotic cells, such as E. coli WCM105, and is secreted into the bacterial culture medium.

In equivalent experiments the presence of full length IgG heavy and light chains in bacterial culture medium is demonstrated for the other constructs generated in Example 1.

### REFERENCES

Simmons L C, Reilly D, Klimowski L, Raju T S, Meng G, Sims P, Hong K, Shields R L, Damico L A, Rancatore P, Yansura D G.
Expression of full-length immunoglobulins in Escherichia coli: rapid and efficient production of aglycosylated antibodies.
J Immunol Methods. 2002 May 1; 263(1-2):133-47.

1. A method for the production of an immunoglobulin or a functional fragment thereof in a prokaryotic host cell, said method comprising:
a. transforming said host cell with (a) a first nucleic acid molecule comprising a nucleic acid sequence encoding a V_{L} and a C_{L} region and
(b) a second nucleic acid molecule comprising a nucleic acid sequence encoding a V_{H}, a C_{H1}, a C_{H2} and at least a portion of a C_{H3} region, wherein said host cell is within culture medium;
b. culturing said host cell under conditions so as to allow said host cell (a) to express (1) said V_{L} and a C_{L} region and (2) said V_{H}, said C_{H1}, said C_{H2} and said portion of said C_{H3} region, and (b) to secrete (a)(1) and (a)(2) to the periplasm of said host cell and thereafter to the culture medium of said host cell, wherein (a)(1) and (a)(2) interact to form said immunoglobulin or functional fragment thereof.

2. The method according to claim 1, wherein said heavy chain comprises a V_{H}, a C_{H1}, a C_{H2} and a full-length C_{H3} region.

3. The method according to claim 1, wherein said immunoglobulin is a full-length immunoglobulin.

4. The method according to claim 1, further comprising the step of recovering said immunoglobulin or said functional fragment thereof from the culture medium.

5. The method according to claim 1, wherein said immunoglobulin is an IgG.

6. The method according to claim 5, wherein said IgG is IgG1.

7. The method according to claim 1, wherein one or more of said first and said second nucleic acid molecules further comprises a nucleic acid sequence encoding for a signal sequence.

8. The method according to claim 7, wherein each of said signal sequences is a prokaryotic signal sequences.

9. The method according to claim 8, wherein one or more of said prokaryotic signal sequences is derived from Escherichia coli.

10. The method according to claim 9, wherein said prokaryotic signal sequence is selected from the group consisting of the signal sequences of MalE, LamB, PelB, LivK, TorT, TolB, DsbA, Pac, TorA, PhoA and OmpA.

11. The method according to claim 7, wherein either or both of said signal sequence is N-terminal with respect to the heavy chain and the light chain.

12. The method according to claim 4, further comprising the step of purifying said immunoglobulin or said functional fragment thereof.

13. The method according to claim 1, wherein said first and second nucleic acid molecules are operably linked to the same promoter.

14. The method according to claim 1, wherein said first and second nucleic acid molecules are not operably linked to the same promoter.

15. The method according to claim 1, wherein said first and second nucleic acid molecules are within the same vector.

16. An immunoglobulin or a functional fragment thereof produced according to claim 1.

17. An immunoglobulin or a functional fragment thereof produced according to claim 1, wherein said immunoglobulin or said functional fragment thereof is aglycosylated.

18. The method of claim 1 wherein said immunoglobulin or a functional fragment thereof, wherein said immunoglobulin or said functional fragment thereof is secreted into the culture medium.

19. The method of claim 1, wherein said immunoglobulin or functional fragment thereof comprises a V_{L} and a C_{L} region and a V_{H}, a C_{H1}, a C_{H2} and at least a portion of a C_{H3} region.

20. The method of claim 18, wherein said prokaryotic host cell carries a mutation in at least one protein of the outer membrane.

21. The method of claim 18, wherein said prokaryotic host cell is Escherichia coli.

22. The method of claim 21, wherein said Escherichia coli carries a mutation in the gene minA and/or minB.

23. The method of claim 20, wherein said Escherichia coli is Escherichia coli strain WCM104 or Escherichia coli strain WCM105.
